(19) United States
(12) Patent Application Publication
(10) Pub. No.: US 2004/0078016 A1
(43) Pub. Date: Apr. 22, 2004

(54) Absorbent article with gravity resistant acquisition layer

(76) Inventor: Andrew Baker, Norcross, GA (US)

Correspondence Address: Patrick A. Doody, Esq., Hunton & Williams, Suite 1200, 1900 K Street, NW, Washington, DC, 20006-1109, US

(21) Appl. No.: 10/437,447

(22) Filed: May 14, 2003

Related U.S. Application Data
(60) Provisional application No. 60/379,738, filed on May 14, 2002.

(51) Int. Cl. A61F 13/15; A61F 13/20

(52) U.S. Cl. 604/378; 604/367

(57) ABSTRACT

An absorbent garment for daytime and nighttime use is disclosed having a moisture impervious outer layer, a moisture pervious inner layer, and an absorbent layer disposed between the inner and outer layers. The absorbent garment also includes an acquisition layer disposed between the inner layer and the storage layer, where the acquisition layer has a fluid drain rate such that it loses less than 55% of its fluid after 60 seconds.

### CROSS REFERENCE TO RELATED APPLICATION

This application claims the benefit of U.S. Provisional Application Serial No. 60/379,738, filed May 14, 2002, the contents of which are incorporated herein by reference in their entirety.

### BACKGROUND OF THE INVENTION

1. Field of the Invention

This invention relates to the field of absorbent garments in general, and more particularly to an absorbent article designed for daytime and nighttime use having an acquisition layer that resists to some degree vertical fluid flow caused by gravitational forces, and that loses less than 55% of its stored fluid after 60 seconds, when subjected to the fluid drain rate test.

2. Description of Related Art

Disposable absorbent articles typically include a moisture-impervious backing sheet, an absorbent pad, and a liner sheet that contacts the body of a person wearing the article. In addition, elasticized regions are provided around the edges of the article to secure the article about the waist and legs of a wearer. Absorbent articles such as diapers typically further comprise opposed front and rear waist portions defining a waist opening, a crotch portion disposed there between, and a pair of elastically contractible leg openings along the side edges of the crotch portion. Disposable diapers having elasticized margins for placement about the legs of a wearer are disclosed in U.S. Pat. No. 4,050,462 and U.S. Pat. No. 5,092,861, the disclosures of which are incorporated by reference herein in their entirety. An absorbent article having elasticized side margins and waist band margins are described in U.S. Pat. No. 4,300,562, the disclosure of which is incorporated by reference herein in their entirety.

Despite previous advancements in the field of absorbent articles, there still is a current need to provide absorbent articles that are better able to contain urinary and fecal excretions. For instance, problems with prior diaper designs include inferior absorbency and leakage of urinary or fecal material from the article. Prolonged contact of liquid or semi-solid excreta with the skin of the wearer also is a continuing problem. For example, the moisture vapor and heat generated by the body exudates trapped within a diaper may lead to conditions adjacent to the wearer's skin that promotes skin irritation, infection, and the like.

Developing highly absorbent articles for use as disposable diapers, adult incontinence pads and briefs, and catamenial products such as sanitary napkins typically entails developing relatively absorbent cores or structures that can acquire, distribute and store large quantities of discharged body fluids, in particular urine. For example, absorbent structures include particulate absorbent polymers often referred to as “hydrogels,” “superabsorbents” or “hydrocolloid” materials. See, for example, U.S. Pat. No. 3,699,103, and U.S. Pat. No. 3,770,731 that disclose the use of such particulate absorbent polymers in absorbent articles. Indeed, the development of high performance diapers has been due in part to thinner absorbent cores that take advantage of the ability of these particulate absorbent polymers to absorb large quantities of discharged aqueous body fluids, typically when used in combination with a fibrous matrix. See, for example, U.S. Pat. No. 4,673,402 and U.S. Pat. No. 4,935,022, that disclose dual-layer core structures comprising a fibrous matrix and particulate absorbent polymers useful in fashioning high performance diapers.

Other absorbent materials capable of providing good absorbency and good capillary fluid transport are polymeric foams. Certain types of polymeric foams have been used in absorbent articles for the purpose of imbibing, and/or wicking aqueous body fluids. See, for example, U.S. Pat. No. 3,563,243 (absorbent pad for diapers and the like where the primary absorbent is a hydrophilic polyurethane foam sheet); U.S. Pat. No. 4,554,297 (body fluid absorbing cellular polymers that can be used in diapers or catamenial products); U.S. Pat. No. 4,740,520 (absorbent composite structure such as diapers, feminine care products and the like that contain sponge absorbents made from certain types of super-wicking, crosslinked polyurethane foams).

There are a number of documents describing absorbent foams that have been made from High internal Phase Emulsions (hereafter referred to as “HIPE”), or hydrophilic, flexible, open-celled foam such as a melamine-formaldehyde foam (e.g., BASOTECT™ made by BASF). See, for example, U.S. Pat. Nos. 5,147,345, 5,260,345, 5,268,224, 5,318,554, 5,331,015, 5,352,711, 5,550,167, 5,632,737, 5,692,939,5,786,395, and 5,851,648. These absorbent HIPE foams provide desirable fluid handling properties, including: (a) relatively good wicking and fluid distribution characteristics to transport the imbibed urine or other body fluid away from the initial impingement zone and into the unused balance of the foam structure to allow for subsequent gushes of fluid to be accommodated; and (b) a relatively high storage capacity with a relatively high fluid capacity under load, i.e. under compressive forces. These HIPE absorbent foams are also sufficiently flexible and soft so as to provide a high degree of comfort to the wearer of the absorbent article; some can be made relatively thin until subsequently wetted by the absorbed body fluid.

U.S. Pat. No. 5,147,345, and 5,310,554 to Young et al. discloses absorbent articles, such as diapers, for the management of incontinence. Such articles utilize in their absorbent cores a fluid acquisition/distribution component and a fluid storage/redistribution component maintained in fluid communication with the acquisition/distribution component. The fluid acquisition/distribution component can be any porous hydrophilic, e.g., fibrous or foam-based, material that will provide an initial Fluid Acquisition Rate of at least 2 mL of synthetic urine per second and will also preferably provide a 30-minute Vertical Wicking Height of at least 2 cm. The fluid storage/redistribution component comprises a hydrophilic, flexible, open-celled polymeric foam having a free absorbent capacity of at least about 12 mL of synthetic urine per gram of dry foam and an absorbent capacity under a 5.1 kPa confining pressure which is at least 5% of this free capacity. Preferred fluid acquisition/distribution component materials comprise chemically stiffened, twisted, curled cellulosic fibers. Preferred fluid storage/redistribution component materials comprise absorbent foams prepared by polymerizing a high internal phase emulsion (HIPE).

U.S. Pat. Nos. 5,268,224, and 5,331,015 to Des Marais et al. discloses absorbent foam materials suitable for use as or in the absorbent cores of absorbent articles, such as diapers that absorb and retain aqueous body fluids. Such foam materials comprise hydrophilic, flexible open-celled structures which are preferably prepared by polymerizing high internal phase (HIPE) water-in-oil emulsions. Such foam materials have a pore volume of from about 12 to 100 mL/g, and a capillary suction specific surface area of from about 0.5 to 5.0 m²/g. These materials also exhibit a resistance to compression deflection such that a confining pressure of 5.1 kPa produces after 15 minutes a strain of from about 5% to 95% compression when the material is saturated at 37° Celsius to its free absorbent capacity with synthetic urine.

U.S. Pat. Nos. 5,851,648, 5,786,396, 5,632,737, 5,692,939, and 5,550,167 disclose absorbent foams materials that are capable of acquiring and distributing aqueous fluids, especially discharged body fluids such as urine. These absorbent foams combine relatively high capillary absorption pressures and capacity-per-weight properties that allow them to acquire fluid, with or without the aid of gravity. These absorbent foams are alleged to give up this fluid to higher absorption pressure storage materials, including foam-based absorbent fluid storage components, without collapsing. These absorbent foams are made by polymerizing high internal phase emulsions (HIPEs).

U.S. Pat. No. 5,352,711 to Des Marais discloses that normally hydrophobic foams and polymerized water-in-oil emulsion foams are rendered hydrophilic by means of treatment with simple surfactants and hydrophilizing agent salts. Thus, a surfactant-containing foam is treated with a solution of, for example, calcium chloride, and is dried to leave a substantially uniformly distributed residue of hydrated or hydratable calcium chloride on the surfactant-containing internal foam surfaces. In-use, the combination of surfactant and calcium chloride hydrate provides a hydrophilic surface to the foam. Other hydratable calcium or magnesium salts such as magnesium chloride can be used. The resulting hydrophilized foams are said to be suitable for use in absorbent devices, including diapers, sanitary napkins, bandages, and the like.

As is apparent from the foregoing, each of these documents presents a variety of mechanisms for improving absorbency in absorbent garments. However, all of these proposed mechanisms are deficient in terms of effectiveness, low product quality, mechanical complexity in design, and/or associated cost inefficiencies.

Polyurethane foams are known to be useful as absorbent materials. For example, U.S. Pat. No. 5,164,421 discloses a hydrophilic foam that is applicable for the manufacture of absorptive devices. The hydrophilic polyurethane foams can be made from aromatic polyisocyanates. Polyurethane foams made from such aromatic polyisocyanates tend to yellow when exposed to light, which is undesirable in a sanitation product or other absorbent articles. In addition, many aromatic polyisocyanates contain undesirable and hazardous chemical components.

Many of the aforementioned garments are designed to be used in caring for infants. Infants of all ages tend to void more often during the day than during the night. For example, ten to eighteen month old infants may void three to four times as often during the day than they do during the night. For this reason, most absorbent garments are designed for daytime use. Absorbent cores of conventional daytime garments are designed primarily for use in an upright position, such as when the user is standing or sitting. These designs recognize that fluids tend to flow vertically into the crotch region of the article due to gravitational force. Operating on the proposition that the absorbent material should be placed where the fluid naturally tends to settle, the absorbent core material in such daytime garments generally is located where it will be most useful to handle the natural flow of fluids. Such construction reduces the amount of unused absorbent, thereby improving comfort and reducing costs.

Conventional diapers use “zoned” absorbent cores that locate the majority of the absorbent material in the crotch and front waist region. In use, urine typically strikes the front waist region of a conventional garment, and then flows downward into the crotch because it can not be absorbed by the portion of the core in the front waist region quickly enough. In order to redistribute the urine that settles in the crotch, and thus improve the efficiency of the garment, manufacturers typically provide the waist regions with materials that promote vertical wicking of fluids away from the crotch, such as a fluid handling layer or a top sheet with the fabric having a machine direction oriented from the crotch to the waist region. Exemplary materials used for this purpose are non-woven carded materials having the fibers oriented in the machine direction of the fabric causing fluids to preferentially travel in the direction of the fibers. Such a solution is incomplete, however, because it fails to provide immediate fluid absorption, and the efficiency of the garment may be reduced because the wicking layer may not be able to redistribute all of the unabsorbed fluid from the crotch region.

Although infants generally void more often during the day, the capacity of a nighttime garment must be greater than that of a daytime product. This is because diapers typically are changed more often during the day than at night, making nighttime use more stressful on the garment. Nighttime voids generally occur while the infant is in a horizontal position. In such a position, gravity draws the fluids towards the waist of the diaper, instead of the crotch where the absorbent is normally concentrated. The capacity of a nighttime product therefore preferably is greater than the capacity of a daytime product.

For these and other reasons, daytime garments are not particularly suitable for nighttime use. One possible solution to this problem would be to manufacture separate daytime and nighttime diapers. This solution would be expensive, however, because it would require separate manufacturing processes. In addition, this would further complicate a parent's purchasing and managing of diaper supplies. A better solution is to provide a single universal diaper that is suitable for both daytime and nighttime use.

A universal diaper could be made by simply adding absorbent core material to the waist regions of a conventional daytime garment. This would be expensive, however, because the material cost for the additional absorbent would increase substantially. In addition, the absorbent material in the waist would see very little usage during the day because the fluid would flow to the crotch region before the absorbent in the waist has time to absorb it. For the same reasons, the absorbent in the crotch would see limited use during the night. The additional absorbent would also make the garment heavier and bulkier, which would render the garment less comfortable.

The description herein of the various known products, methods, and apparatus and their attendant disadvantages is in no way intended to limit the scope of the present invention, or to imply that the present invention does not include some or all of the various elements of the products, methods, and apparatus in one form or another. Indeed, various embodiments of the invention may be capable of overcoming some of the disadvantages noted herein, while still retaining some or all of the various elements of the known products, methods, and apparatus in one form or another.

All documents described herein are incorporated by reference in this disclosure in their entirety.

### SUMMARY OF THE INVENTION

There is a need to provide an absorbent garment designed for daytime and nighttime use that does not require the addition of a substantial amount of absorbent material, that does not cost substantially more than a conventional garment to produce, and that is as comfortable as a conventional garment. These and other needs are addressed by various embodiments of the invention.

It is therefore a feature of an embodiment of the invention to provide an absorbent article that is capable of both day and nighttime use. It is an additional feature of the invention to provide an absorbent article that can acquire and absorb fluids quickly, and then hold the fluid without much gravitational effect until the storage layer can fully absorb it. This feature of the invention enables the absorbent article to adequately acquire the fluid, and then distribute it efficiently to the absorbent core storage layer.

In accordance with these and other features of embodiments of the invention, there is provided a disposable absorbent garment having an absorbent core designed for both daytime and nighttime use. The absorbent core may have two layers: (i) a storage layer; and (ii) an acquisition layer. Stated generally, the invention includes:

a liquid impervious outer layer;

a liquid pervious inner layer;

a storage layer disposed between the outer layer and the inner layer; and

an acquisition layer disposed between the inner layer and the storage layer, whereby the acquisition layer has a fluid drain rate of less than about 55% fluid loss after 60 seconds.

In accordance with another feature of embodiments of the invention, there is provided a disposable absorbent garment having:

a liquid impervious outer layer;

a liquid pervious inner layer;

a storage layer disposed between the outer layer and the inner layer; and

an acquisition layer disposed between the inner layer and the storage layer, whereby the absorbent article has an absorbent capacity under load in the front portion thereof of above about 1.1 g/cm², and an absorbent capacity under load in the rear portion thereof of between about 0.5 and about 0.9 g/cm².

Another feature of an embodiment of the invention includes a disposable absorbent garment having:

a liquid impervious outer layer;

a liquid pervious inner layer;

a storage layer disposed between the outer layer and the inner layer; and

an acquisition layer disposed between the inner layer and the storage layer, whereby the acquisition layer has a peak capacity of greater than about 25 grams.

In accordance with an additional feature of an embodiment of the invention, there is provided a method of making an absorbent article that includes:

providing a liquid impervious outer layer;

providing a liquid impervious inner layer;

providing a storage layer;

disposing the storage layer between the inner layer and the outer layer;

providing an acquisition layer; and

disposing the acquisition layer between the inner layer and the storage layer,

wherein the acquisition layer has a fluid drain rate of less than about 55% fluid loss after 60 seconds.

In accordance with an additional feature of an embodiment of the invention, there is provided a method of making an absorbent article that includes:

providing a liquid impervious outer layer;

providing a liquid impervious inner layer;

providing a storage layer;

disposing the storage layer between the inner layer and the outer layer;

providing an acquisition layer; and

disposing the acquisition layer between the inner layer and the storage layer,

whereby the absorbent article has an absorbent capacity under load in the front portion thereof of above about 1.1 g/cm², and an absorbent capacity under load in the rear portion thereof of between about 0.5 and about 0.9 g/cm².

Another embodiment of the invention encompasses a method of making an absorbent article as described above whereby the acquisition layer has a peak capacity of greater than about 25 grams.

One advantage of an embodiment of the invention is that the garment may be used satisfactorily for both daytime and nighttime use. Another advantage of an embodiment of the invention is that the garment does not require significantly more absorbent material than a conventional garment. Yet another advantage of an embodiment of the invention is that the absorbent garment provides a comfortable fit.

These and other objects, features and advantages of the invention will be apparent through the detailed description of the preferred embodiments and the drawings attached hereto. It also is to be understood that both the foregoing general description and the following detailed description are exemplary and explanatory and are not restrictive of the scope of the invention.

### BRIEF DESCRIPTION OF THE DRAWINGS

The invention will be described with respect to the accompanying drawings, in which like elements are referenced with like numbers.

FIG. 1 is a drawing of an embodiment of the present invention, as it appears when worn by a user in a standing or sitting position, with certain parts partially cut away to expose the inside of the embodiment;

FIG. 2 is an exploded view of an embodiment of the present invention with elastic members shown in the elongated position for clarity, the side seams separated, and the garment laid flat;

FIG. 3 is a depiction of an embodiment of a storage layer in the fully flattened position;

FIG. 4 is a depiction of another embodiment of a storage layer 16 in the fully flattened position;

FIG. 5 is a depiction of yet another embodiment of a storage layer in the fully flattened position, showing super absorbent particle density.

FIG. 6 is a depiction of an embodiment of an acquisition layer in the fully flattened position;

FIG. 7 is a cut away side view of an embodiment of the present invention in the upright position, indicating the flow of liquid within the garment;

FIG. 8 is a graph showing the peak capacity of fluid retained by various acquisition layer materials;

FIG. 9 is a graph showing the percent fluid loss after 60 seconds of various acquisition layer materials subjected to a fluid drain rate test;

FIG. 10 is a schematic view of the method used to make by hand some of the absorbent garments used and tested in the examples; and

FIG. 11 is a side view of an absorbent article showing the cutting point made when making by hand some of the absorbent garments used and tested in the examples.

### DETAILED DESCRIPTION OF THE PREFERRED EMBODIMENTS

“Garment” and “absorbent article,” as used herein, are interchangeable terms that refer to articles and garments that absorb and contain body exudates, and, more specifically, refers to articles and garments that are placed against or in proximity to the body of the wearer to absorb and contain the various exudates discharged from the user's body. A non-exhaustive list of examples of absorbent articles and garments includes diapers, diaper covers, disposable diapers, training pants, feminine hygiene products, and adult incontinence products. The invention can be used with any and all of the foregoing classes of absorbent articles and garments, without limitation, whether disposable or otherwise. Furthermore, the invention will be understood to encompass, without limitation, all classes and types of absorbent articles and garments, including those described above. Reference herein to “garment” and “absorbent article” will be understood to include all of the above. In addition, although various embodiments of the invention are described in the context of a diaper, it is readily apparent and understood that this is not intended to limit the invention.

Throughout this description, the expression “fluid drain rate” denotes a characteristic feature of an absorbent article, or component thereof, that enables the article or component to acquire fluid, and then retain the fluid against the force of gravity. The fluid drain rate can be measured by the fluid drain rate test described in more detail in the Testing Methods section below. Stated more generally here, the fluid drain rate test measures the acquisition layer or absorbent material's ability to hold fluid against the pull of gravity, whereby a material containing at least the acquisition layer is saturated with fluid, and then partially removed from the fluid and held vertically for up to about 60 seconds or more to determine how much of the fluid drained out from the material. The total amount of fluid retained by the material also is measured and the maximum amount of fluid retained is referred to herein as the “peak capacity.” Materials retaining more fluid, and consequently, draining less fluid, are desirable for use in an acquisition layer in the invention.

Throughout this description, the expression “substantially no capillary pore orientation” denotes a porous material that does not have oriented pores that provide capillary action. This most preferably occurs when the sum of the capillary force vectors for the porous material is substantially close to zero. Preferably, a material is said to have substantially no capillary pore orientation if it contains less than 5%, more preferably, less than 2.5%, and most preferably, less than 1% of interconnected pores from one side of the material to the other.

Throughout this description, the expressions “upper layer” and “lower layer,” or “outer layer” and “inner layer” that refer to the layers disposed above and/or below the absorbent core of the invention are used merely to describe one layer above the core, and one layer below the core (or one layer outside the core from the wearer, and one layer inside the core from the wearer). The upper or inner layer need not always remain vertically above or inside the core, and the lower or outer layer need not always remain vertically below or outside the core. Indeed, many embodiments of the invention encompass various configurations of an absorbent core that is folded in such a manner that the upper layer ultimately becomes the vertically highest and vertically lowest layer at the same time. Other configurations are contemplated within the context of the present invention. In the present invention, “top sheet” is used synonymously with upper layer, and “back sheet” is used synonymously with lower layer.

Throughout this description, the terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet. The term “component” can refer to, but is not limited to, designated selected regions, such as edges, corners, sides or the like; structural members, such as elastic strips, absorbent pads, stretchable layers or panels, layers of material, or the like; or a graphic.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the expression “tow fibers” relates in general to any continuous fiber. Tow fibers typically are used in the manufacture of staple fibers, and preferably are comprised of synthetic thermoplastic polymers. Usually, numerous filaments are produced by melt extrusion of the molten polymer through a multi-orifice spinneret during manufacture of staple fibers from synthetic thermoplastic polymers in order that reasonably high productivity may be achieved. The groups of filaments from a plurality of spinnerets typically are combined into a tow which then is subjected to a drawing operation to impart the desired physical properties to the filaments comprising the tow.

Throughout this description, the expression “absorbent capacity under load” denotes the ability of an absorbent article to absorb fluid under a given pressure. Specifically, absorbent capacity under load can be measured by the “PAD AUL” test outlined in the Testing Methods section below, and is expressed, unless otherwise noted herein, in terms of grams per square centimeter of material.

The present invention contemplates a disposable absorbent garment having an absorbent core designed for both daytime and nighttime use. The absorbent core has two layers: (i) a storage layer; and (ii) an acquisition layer. Alternatively, the storage layer is the absorbent core material, and the acquisition layer is a separate material. For example, the absorbent core material can comprise the storage layer surrounded by an upper and lower tissue layer, whereby the acquisition layer may be disposed inside our outside the upper and lower tissue layers. The absorbent article of the invention preferably includes:

a liquid impervious outer layer;

a liquid pervious inner layer;

a storage layer disposed between the outer layer and the inner layer; and

an acquisition layer disposed between the inner layer and the storage layer, whereby the acquisition layer has a fluid drain rate of less than about 55% of fluid loss after 60 seconds.

A preferred embodiment of the invention includes an absorbent garment having:

a liquid impervious outer layer;

a liquid pervious inner layer;

a storage layer disposed between the outer layer and the inner layer; and

an acquisition layer disposed between the inner layer and the storage layer, whereby the absorbent article has an absorbent capacity under load in the front portion thereof of greater than about 1.1 g/cm², and an absorbent capacity under load in the rear portion thereof of between about 0.5 to about 0.9 g/cm². Such relatively discrete zones of absorbent capacity permit the use of the garment during the day and the night.

Yet another preferred embodiment of the invention includes an absorbent garment having:

a liquid impervious outer layer;

a liquid pervious inner layer;

a storage layer disposed between the outer layer and the inner layer; and

an acquisition layer disposed between the inner layer and the storage layer, whereby the acquisition layer has a peak capacity greater than about 25 grams.

The features of the acquisition layer described above provide an absorbent article that can acquire fluid quickly from a user, and then hold on to the fluid under the impetus of gravity so that all of the fluid does not drain to the lowest point in the absorbent core. Rather, the ability to hold on to the fluid enables efficient distribution of the fluid throughout the absorbent core, or to portions of the absorbent core that can readily absorb the fluid (e.g., in the case of zoned absorbent cores, the fluid can be distributed to portions of the storage component of the absorbent core that contain more absorbent material than other portions).

Providing an acquisition layer that resists free fluid flow (i.e., gravity resistant) was previously thought to be counterintuitive. Acquisition layers were primarily used to acquire the fluid and transport the fluid to the storage layer, or absorbent core, as quickly as possible. In contrast to conventional wisdom, the present invention provides a fluid acquisition layer that acquires fluid quickly, but then is capable of holding on the fluid against the impetus of gravity to efficiently distribute the fluid to portions of the storage layer, or absorbent core, that can absorb the fluid more readily. By way of example only, and not intending on being bound by any theory of operation, a gravity resistant fluid acquisition layer can provide advantages to an absorbent garment worn during the evening, especially a garment having more absorbent capacity in the front portion thereof than in the rear portion. For example, if a wearer were lying on his or her back and insulted the garment, the fluid typically would flow by gravity to the rear of the garment. It is believed in the present invention that the fluid acquisition layer provides some resistance to this flow by gravity, thereby distributing some of the fluid to the front portion of the garment where it has a greater absorbent capacity.

Another embodiment of the invention includes a method of making an absorbent article that preferably includes:

providing a liquid impervious outer layer;

providing a liquid impervious inner layer;

providing a storage layer;

disposing the storage layer between the inner layer and the outer layer;

providing an acquisition layer; and

disposing the acquisition layer between the inner layer and the storage layer,

wherein the acquisition layer has a fluid drain rate of less than about 55% fluid loss after 60 seconds.

An additional preferred embodiment of the invention encompasses a method of making an absorbent article that includes:

providing a liquid impervious outer layer;

providing a liquid impervious inner layer;

providing a storage layer;

disposing the storage layer between the inner layer and the outer layer;

providing an acquisition layer; and

disposing the acquisition layer between the inner layer and the storage layer,

whereby the absorbent article has an absorbent capacity under load in the front portion thereof of above about 1.1 g/cm², and an absorbent capacity under load in the rear portion thereof of between about 0.5 and about 0.9 g/cm².

Other preferred methods of the invention include those as described above, with the exception that the acquisition layer has a peak capacity of greater than 25 grams. It is preferred that the peak capacity of the acquisition layer be greater than about 30 grams, more preferably greater than about 35 grams, and most preferably greater than about 40 grams.

The absorbent garment preferably has a longitudinal centerline corresponding roughly to the rear-to-front axis of a wearer, a lateral centerline orthogonal to the longitudinal centerline and dividing the garment into front and rear halves. The front and rear halves of the garment correspond to the front of back of the wearer, respectively. The front half of the garment terminates at a front waist edge.

The garment preferably has an outer layer of a substantially liquid impervious material, an inner layer of a substantially liquid pervious material overlaying and operatively associated with the outer layer, and an absorbent core disposed between the outer layer and inner layer. The absorbent core, in turn, preferably has a storage layer and an acquisition layer.

The storage layer preferably, although not necessarily, is disposed closer to the outer layer of the garment than the acquisition layer. The acquisition layer therefore preferably is disposed closer to the inner layer of the garment than the storage layer. The storage layer preferably has longitudinally opposed front storage layer and rear storage layer edges with a central storage layer region between the front and rear edges. The front storage layer edge is located between the rear storage layer edge and the front waist edge of the garment. The storage layer preferably has a storage layer thickness that generally is orthogonal to the outer layer of the garment.

The acquisition layer preferably has longitudinally opposed front acquisition layer and rear acquisition layer edges with a central storage layer region between them. The front acquisition layer edge is located between the rear acquisition layer edge and the front waist edge of the garment. The acquisition layer has an acquisition layer thickness that generally is orthogonal to the outer layer of the garment.

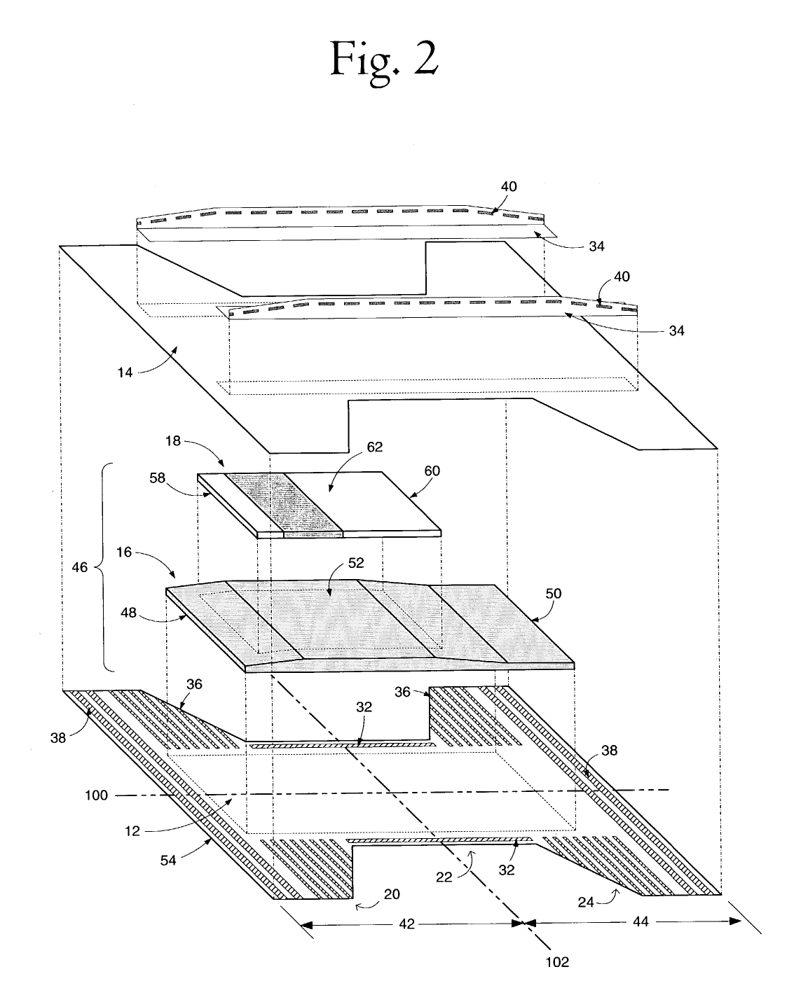

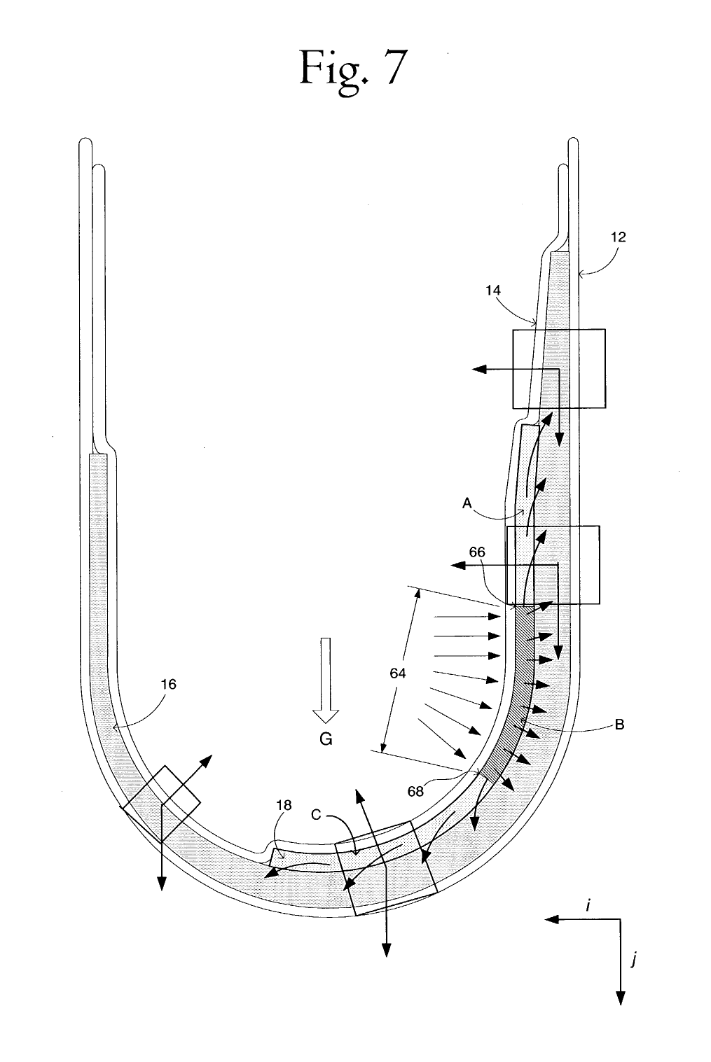

In one preferred embodiment of the invention, the absorbent capacity of the absorbent article varies throughout its longitudinal dimension. In another embodiment, more than half of the absorbent capacity of the absorbent article is located in the front half of the garment. The front portion and rear portion of an absorbent garment are determined by folding the garment about its lateral centerline (as shown, for example, in FIG. 7), where the lateral centerline equals zero, positive longitudinal distances are in the front half 42 (FIG. 2) of the garment, and negative longitudinal distances are in the rear half 44 of the garment. It is preferred in the present invention that the absorbent capacity under load values for the front portion of the garment be taken at a distance within the range of from about 2.5 to about 7 cm from the centerline, more preferably from about 3.5 to about 6 cm, and most preferably at or about 4.5 cm from the centerline. It is preferred in the present invention that the absorbent capacity under load values for the rear portion of the garment be taken at a distance within the range of from about −1 to about −6 cm from the centerline, more preferably from about −2 to about −4.5 cm, and most preferably at or about −3.0 cm from the centerline.

In another preferred embodiment of the invention, the acquisition layer is comprised of a foam material having substantially no capillary pore orientation, and more preferably, a foam that has a low density, including, inter alia, an open cell polyurethane foam, and most preferably, a low density, open-cell polyurethane foam derived from an aliphatic isocyanate. In another preferred embodiment of the invention the acquisition layer thickness varies. In still another preferred embodiment of the invention, the acquisition layer has several different zones made from different materials. In yet another preferred embodiment, at least half of the acquisition layer is located in the front half of the garment.

The inner layer and outer layer each preferably has greater longitudinal and lateral dimensions than both the storage layer and the acquisition layer, such that the inner and outer layers fully envelop the absorbent core.

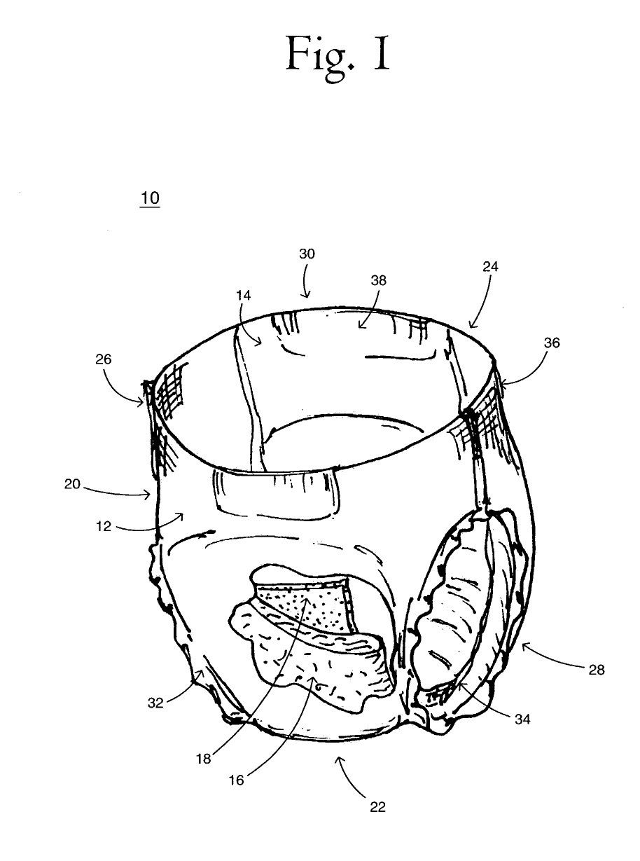

FIG. 1 illustrates an embodiment of a garment 10 of the present invention as it appears when worn by a user in a standing or sitting position with the back sheet 12 and storage layer 16 partially cut away to show the interior of the garment 10. The garment 10 preferably is comprised of an exterior facing moisture impervious outer layer or “back sheet” 12, and a moisture pervious body-contacting inner layer or “top sheet” 14. An absorbent core comprising primary storage layer 16, which may or may not additionally include an acquisition layer 18, is disposed between the top sheet 14 and the back sheet 12, with the acquisition layer 18 preferably being between the storage layer 16 and the top sheet 14. The precise positioning of the storage layer 16 and acquisition layer 18 is not critical to the invention, and the acquisition layer 18 may be disposed between the storage layer 16 and the back sheet 12. The garment typically will have a front waist region 20 that corresponds with the front of the user, a crotch region 22 and a rear waist region 24.

Any number of construction processes or techniques may be used to fabricate the garment of the present invention. The garment 10 depicted in FIG. 1 may be constructed by assembling the various parts in a flat position, then bringing the front and rear waist regions 20, 24 together and joining them to form side seams 26 and leaving regions unjoined to form leg holes 28 and a waist hole 30. Garments of this design are often called “pull-on” garments. In another embodiment, the side edges may be left unjoined during the construction process and instead provided with fastening mechanisms so that the garment 10 may attached in the waist regions by the user, who can adjust the fit of the garment 10. Although the present invention is herein described in terms of a pull-on garment, it is readily apparent and understood that the present invention is applicable to other garment types, and is not limited to pull-on garments.

The garment of the present invention also may be constructed in any number of steps and from any number of subassemblies. In one embodiment, the garment may be constructed using one continuous piece of material for each of the top sheet 14 and back sheet 12. In another embodiment, the invention is made from separate front and rear subassemblies that are made from separate pieces of top sheet and back sheet material, then joined together along one or more seams. Other combinations of subassemblies or construction steps also may be used.

The various parts of the garment 10 are operatively associated with one another in such a manner that the garment will maintain its desired structure during use. The parts may be operatively associated by a variety of mechanisms known in the art, including, but not limited to: using hot melt adhesives, construction adhesives, ultrasonic welding or stitching. All of the parts may be joined to each adjacent part, or some parts may not be joined to others. In one embodiment, the top sheet 14 and back sheet 12 are bonded to one another around their perimeter regions, thereby encasing and holding the storage layer 16 and acquisition layer 18 between them without having to join the storage layer 16 or the acquisition layer 18 to other parts. The top sheet or back sheet also may be operatively associated with the storage layer 16 or acquisition layer 18. As understood herein, the term “operatively associated” includes directly joining one part to another, indirectly joining parts together through one or more intermediary parts, whether those intermediary parts are described herein or not, joining parts in such a manner that unjoined parts are captured or held in their proper place, and any other suitable joining mechanisms.

Still referring to FIG. 1, the garment may include various mechanisms for improving the fit of the garment to the wearer. In one embodiment of the invention, leg gathers 32 are used to contract the leg holes 28 of the garment 10 around the wearer's legs to prevent body waste from escaping. In one embodiment, leg gathers 32 are incorporated into the garment 10 by extending one or more pieces of elastic and bonding them to the top sheet 14, back sheet 12, or other components while in the elongated state. When the elastic contracts, the leg holes 28 constrict around the wearers legs.

The elastic elements may be made from natural rubber, lycra, polyurethane, heat shrinkable polymer ribbons, or any other suitable elastic material or composite. Leg gathers are known in the art, and U.S. Pat. No. 5,660,664 issued to Hermann, which is incorporated herein by reference in its entirety, discloses an exemplary method of manufacturing absorbent articles containing leg gathers that is suitable for the present invention.

In another embodiment of the invention the garment 10 is equipped with standing leg gathers 34 (FIGS. 1 and 2). Standing leg gathers 34 generally include flaps or sleeves of liquid impervious material that have one or more elastic elements 40 in them. The flaps are drawn against the user's body by the force of the elastic, much like the leg gathers 32 described herein, and help seal the garment 10 against leakage. The elastic elements 40 may be made from any suitable elastic material, and the flap portions of the leg gathers 32 may be made as an integral part of the top sheet 14 or back sheet 12, or from additional fabric material. Standing gathers suitable for use in the present invention are disclosed, for example, in U.S. Pat. No. 5,292,316 issued to Suzuki, which is incorporated herein by reference in its entirety, in a manner consistent with the present invention.

In yet another embodiment of the present invention, the garment depicted in FIG. 2 has side elastics 36 to help the garment conform to the wearer's waist. The side elastics 36 may be integrated into the garment 10 in much the same manner as the leg gathers 32, as described herein, or by any other suitable means. Side elastics 36 conform the garment to the user's waist to prevent leakage and to improve the comfort and fit of the garment 10. The side elastics may stretch across the entire width of the garment 10, or they may extend only across particular regions of the garment. Side elastics are disclosed in more detail in U.S. patent application Ser. No. 08/931,389, filed on Sep. 16, 1997, which is incorporated herein by reference in its entirety, and in a manner consistent with the present invention.

In a preferred embodiment of the invention, additional features may be added to the garment 10 around the waist hole 30 to improve fit, comfort, and leakage prevention and to provide other benefits. In one embodiment, waist elastics 38 are added to the front and rear waist regions 20, 24 near the edges that form the perimeter of the waist hole 30. The waist elastics may be an elastic foam or any other suitable elastic material, examples of which are provided elsewhere herein, and the waist elastics may extend across the entire garment 10 or only partly across the garment 10. Examples of such features are disclosed in U.S. Pat. No. 5,034,008 issued to Breitkopf and U.S. Pat. No. 5,435,806 issued to Daugan et al., the disclosures of which are incorporated herein by reference in its entirety, and in a manner consistent with the present invention.

FIG. 2 is an exploded view of an embodiment of the present invention with elastic members shown in the elongated position for clarity, the side seams separated, and the garment laid flat. The garment 10 has a longitudinal centerline 100 corresponding roughly to the rear-to-front axis of the wearer, and a lateral centerline 102, orthogonal to the longitudinal centerline 100, and corresponding roughly to the side-to-side axis of the wearer. The lateral centerline 102 divides the garment into a front half 42 and a rear half 44, which correspond with the front and rear of the wearer, respectively. With the garment 10 laid flat, the front waist region 20 lies entirely within the front half 42, and the rear waist region 24 lies entirely within the rear half 44, although skilled artisans will recognize that the front or rear waist regions (20, 24) may extend beyond the front and rear halves (42, 44), respectively, of the garment 10. A crotch region 22 joins the front waist region 20 and the rear waist region 24. The garment 10 has a front waist edge 54 at the longitudinally foremost part of the garment 10, which forms the front half of the waist hole 30 (see FIG. 1) when the garment is worn. As understood herein, the “front” or “forward limit” of any part of the garment refers to the portion of that part that lies farthest towards the front waist edge 54 of the garment, and the “rear” or “rearward limit” corresponds to the portion of the part that is located farthest from the front waist edge 54. Those skilled in the art will appreciate, however, that the terms “front” and “rear” are used herein for purpose of illustration, and that the garment 10 of the invention may be reversed.

The back sheet or outer layer 12 preferably is made from a substantially liquid impervious material. The selection and manufacture of such materials is well known in the art, and is disclosed, for example, in U.S. Pat. No. 6,123,694 issued to Peniak et al., and U.S. Pat. No. 6,176,952 issued to Maugans et al., the disclosures of each are incorporated herein by reference in their entirety, and in a manner consistent with the present invention. In one embodiment, the back sheet 12 is made from a thin thermoplastic material, such as a pigmented polyethylene film having a thickness in the range of 0.02-0.04 mm. The back sheet 12 also may be a layered material comprised of one or more layers of melt blown polypropylene or melt blown polyethylene, sandwiched between layers of spun-bonded material. Additional layers may be added to the back sheet 12 in order to provide the back sheet 12 with other desirable properties, such as to improve the tactile feel or “hand” of the back sheet. The back sheet may also be entirely or partly gas pervious to allow the garment to circulate air, or “breathe.” In one embodiment of the invention, the back sheet 12 is made from several pieces of material joined at or near their edges with little or no overlap, which may have dissimilar physical properties.

In one embodiment, the back sheet 12 defines the outer perimeter of the garment 10, such that none of the other parts of the garment 10 extend beyond the outline of the back sheet 12 when the side seams 26 (see FIG. 1) are separated and the various parts are laid flat, as the embodiment is depicted in FIG. 2. However, in other embodiments the back sheet 12 may not define the outer perimeter of the garment, and other parts may extend beyond the edges of the back sheet 12.

The top sheet or inner layer 14, which overlays the back sheet 12, preferably is made from a substantially liquid pervious material. The top sheet 14 may typically be made of a carded polyester fiber with a latex binder, or of a spun-bonded polypropylene having continuous fibers and thermally bonded by patterned calendar rolls. The top sheet 14 may be treated over its entire surface to render it hydrophilic. The top sheet 14 also may be zone-treated with a surfactant to make it hydrophilic only in certain target areas. The surface treatment can be accomplished by a variety of means well known in the art, and the treatment may include the addition of skin-wellness ingredients such as Vitamin E and aloe vera. In one embodiment, the top sheet 14 is made from a laminate of various layers of material. In another embodiment, the top sheet 14 is made from several pieces of material joined at or near their edges with little or no overlap, which may have dissimilar physical properties. Such an embodiment is disclosed, for example, in U.S. Pat. No. 5,275,590 issued to Huffman, et al., the disclosure of which is incorporated herein by reference in its entirety, and in a manner consistent with the present invention.

In the embodiment of the invention depicted in FIG. 2, the top sheet 14 has substantially the same planar dimensions as the back sheet 12, such that the perimeter of the top sheet 14 matches the perimeter of the back sheet 12. In other embodiments, the top sheet may be larger or smaller than the back sheet, and may have a different general shape. In one embodiment, the top sheet 14 is large enough to cover all of the parts of the garment that are sandwiched between the top sheet 14 and the back sheet 12, such as the waist elastics 38, side elastics 36, leg gathers 32, storage layer 16, and acquisition layer 18.

In one embodiment of the present invention, the top sheet 14 is operatively associated with the back sheet 12 around the perimeter of the top sheet 14. In this embodiment, the top sheet 14 and back sheet 12 may be operatively associated with one another by use of hot melt adhesives, ultrasonic bonding, or other means known in the art. Also in this embodiment, the top sheet 14 and back sheet 12 may be bonded to one another in substantially all areas not having intermediately placed parts, such that the intermediately placed, or “sandwiched,” parts are physically captured between the top sheet 14 and back sheet 12.

An absorbent core 46, preferably comprising a storage layer 16 and an acquisition layer 18, is disposed between the back sheet 12 and the top sheet 14. The acquisition layer 18 preferably is located between the top sheet 14 and the storage layer 16 although other layers or materials may be disposed between them. Other parts, such as a liquid permeable tissue (not shown), may be located between the acquisition layer 18 and the storage layer 16, or between the top sheet 14 or back sheet 12 and the various parts comprising the absorbent core 46.

The storage layer 16 may be made from any absorbent material or materials known in the art, such as tow fibers, wood fibers or other fibers such as chemical wood pulp, or any other suitable liquid absorbing material such as commercially available fluff pulp or fluffed bleached Kraft softwood pulp. In one embodiment of the invention, the storage layer 16 comprises a combination of a porous fibrous web and super absorbent particles. Such storage layers are known in the art and are disclosed, for example, in U.S. Pat. Nos. 5,281,207 and 6,068,620 issued to Chmielewski et al., the disclosures of which are incorporated herein by reference in its entirety, and in a manner consistent with the present invention. In such an embodiment, the storage layer 16 may be surrounded by a liquid pervious tissue over-wrap, or other material.

It is preferred in the invention that storage layer 16 include at least a fibrous component and an absorbent component. The fibrous component of the storage layer 16 preferably is comprised of hard or soft wood pulps, such as Kraft pulp, conventional fluff and/or tow fibers, such as a crimped tow of cellulose acetate or polyester. Before making a storage layer 16 that includes a tow fiber, the tow fiber typically is unwound and opened, and then cut at various lengths to provide a fibrous mass of material. Skilled artisans are aware of techniques available to open tow fibers and form the opened fibers into a fibrous mass. Optionally, it is advantageous to introduce from about 1-5% of a thermally bondable fiber into the fibrous component of the storage layer 16 for wet strength and core stability in use.

Certain fibrous and particulate additives preferably are used as constituent elements of the storage layer 16, in addition to the fibrous component and absorbent component. Particulate additives may be added to storage layer 16 in addition to or as a substitute for the foregoing fibrous additives in order to maintain high SAP efficiency. The particulate additives preferably are insoluble, hydrophilic polymers with particle diameters of 100 μm or less. The particulate additives are chosen to impart optimal separation of the SAP particles. Examples of preferred particulate additive materials include, but are not limited to, potato, corn, wheat, and rice starches. Partially cooked or chemically modified (i.e., modifying hydrophobicity, hydrophilicity, softness, and hardness) starches can also be effective. Most preferably, the particulate additives comprise partially cooked corn or wheat starch because in this state, the corn or wheat are rendered larger than uncooked starch and even in the cooked state remain harder than even swollen SAP. In any event, regardless of the particulate additive chosen, one of the many important criteria is to use particulate additives that are hard hydrophilic materials relative to swollen SAP or which are organic or inorganic polymeric materials about 100 microns in diameter. Fibrous and particulate additives can be used together in these absorbent laminates. Examples of SAP/particulate and SAP/fiber/particulate additives include those described in, for example, U.S. Pat. No. 6,068,620.

Any superabsorbent polymer (SAP) now known or later discovered may be used in storage layer 16 so long as it is capable of absorbing liquids. Useful SAP materials are those that generally are water-insoluble but water-swellable polymeric substance capable of absorbing water in an amount that is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Examples of suitable SAP are water swellable polymers of water soluble acrylic or vinyl monomers crosslinked with a polyfunctional reactant. Also included are starch modified polyacrylic acids and hydrolyzed polyacrylonitrile and their alkali metal salts. A more detailed recitation of superabsorbent polymers is found in U.S. Pat. No. 4,990,541 to Nielsen, the disclosure of which is incorporated herein by reference in its entirety.

Commercially available SAPs include a starch modified superabsorbent polymer available under the tradename SANWET® from Hoechst Celanese Corporation, Portsmouth, Va. SANWET® is a starch grafted polyacrylate sodium salt. Other commercially available SAPs include a superabsorbent derived from polypropenoic acid, available under the tradename DRYTECH® 520 SUPERABSORBENT POLYMER from The Dow Chemical Company, Midland Mich.; AQUA KEEP manufactured by Seitetsu Kagaku Co., Ltd.; ARASORB manufactured by Arakawa Chemical (U.S.A.) Inc.; ARIDALL 1125 manufactured by Chemdall Corporation; FAVOR manufactured by Stockhausen Inc.; HYSORB, available from BASF Aktiengesellschaft, Ludwigshafen, Germany; AQUA KEEP SA60S, manufactured by Seitetsu Kagaku Co., Ltd.; DIAWET, commercially available from Mitsubishi Chemicals, Japan; FLOSORB, available from SNF Floerger, France, AQUALIC, available from Nippon Shokubai, Osaka, Japan.

The storage layer 16 preferably is generally elongated along the longitudinal axis 100 of the garment, and may extend along either or both of the lateral and longitudinal axes 102, 100 to the outer perimeter of the garment. In the embodiment depicted in FIG. 2, the storage layer 16 is substantially rectangular in shape, however, it may also have rounded ends or other shapes, such as an “I” shape or a “T” shape.

The storage layer 16 may have a front storage layer edge 48 defining the forward limit of the storage layer 16, a rear storage layer edge 50 defining the rearward limit of the storage layer 16, and a central storage layer region 52 located between the front and rear storage layer edges 48, 50. Other configurations of storage layer 16 are encompassed by the present invention.

In one embodiment of the present invention, the majority of the storage layer's 16 absorbent capacity is “zoned” to be greater in the front half 42 of the garment 10. The absorbent capacity of a storage layer 16 generally is measured as the volume of fluid the storage layer 16 can retain in a given area of the layer, and typically is expressed as a measure of grams per square centimeter. Zoned storage layers 16 generally are known in the art.

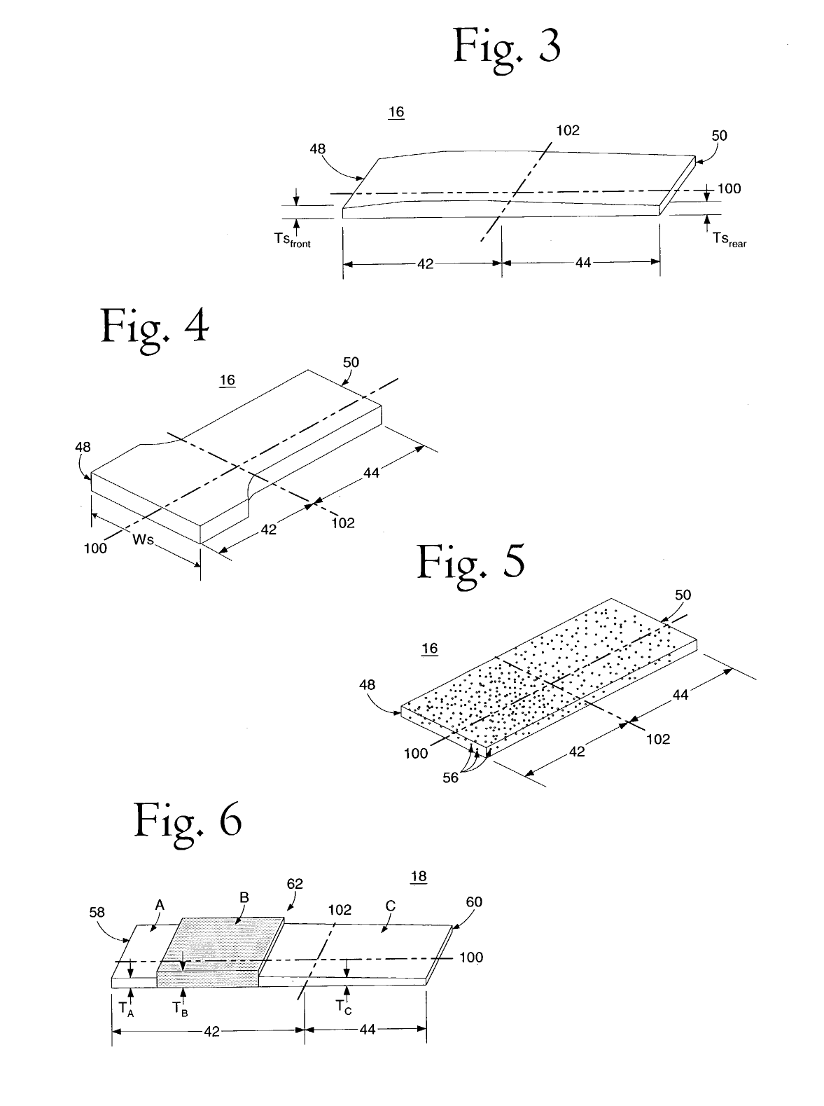

There are numerous methods for providing a greater amount of absorbent capacity in the front half 42 of the garment 10. For example, a greater physical volume of the storage layer 16 may be located in the front half 42 by positioning the storage layer 16 closer to the front edge 54. A greater physical volume of the storage layer 16 may also be provided in the front half 42 by making the storage layer 16 thicker in the front half 42, as is shown in FIG. 3, or wider in the front half 42, as is shown in FIG. 4. Furthermore, in an embodiment in which the absorbent core 16 comprises super absorbent particles 56, the absorbent capacity may be varied by providing a greater or lesser density of super absorbent particles 56 in the front or half 42, as is shown in FIG. 5. There are other ways to increase or decrease the absorbent capacity of the storage layer 16, such as by varying the basis weight or free volume of the storage layer 16 in the front half 42 or the back half 44. The above listed methods for varying a storage layer's absorbent capacity are meant to be exemplary, and the present invention is not intended to be limited to any one method of varying the absorbent capacity of a storage layer. These, and other methods known in the art, may be applied by one skilled in the art to practice the present invention without undue experimentation.

In one embodiment, 51% to about 90% of the storage layer's 10 absorbent capacity is in the front half 42 of the garment. In a more preferred embodiment, about 60% to about 80% of the storage layer's 10 absorbent capacity is in the front half 42 of the garment. In a most preferred embodiment, about 65% of the storage layer's 16 absorbent capacity is in the front half 42 of the garment.

It is preferred in the present invention that the absorbent garment posses an absorbent capacity under load in the front portion thereof of greater than about 1.1 g/cm², and an absorbent capacity under load in the rear portion thereof within the range of from about 0.5 to about 0.9 g/cm². The absorbent capacity under load is measured in accordance with the PAD AUL test described in the Testing Methods section below, and the samples for the “front” and “rear” absorbent capacity under load measurements are taken from the front and rear portions of the garment, as defined above. It is preferred in certain embodiments of the invention that the absorbent capacity under load in the front portion be within the range of from about 1.2 to about 2.0 g/cm², and more preferably from about 1.25 to about 1.5 g/cm² and most preferably about 1.3 to about 1.4 g/cm². It is preferred in certain embodiments of the invention that the absorbent capacity under load in the rear portion be within the range of from about 0.55 to about 0.8 g/cm², and more preferably from about 0.6 to about 0.75 g/cm².

Referring again to FIG. 2, the absorbent core 46 of the garment 10 also preferably comprises an acquisition layer 18 disposed between the storage layer 16 and the top sheet 14. In one embodiment, at least half of the acquisition layer 18 is located in the front half 42 of garment 10, although this configuration is not required in the invention.

The acquisition layer 18 generally is elongated along the longitudinal axis 100 of the garment, and may extend along either or both of the lateral and longitudinal axes 102, 100 to the outer perimeter of the garment. The acquisition layer 18 generally is sized and located to overlap the storage layer 16, however, some parts of the acquisition layer 18 may extend beyond the edges of the storage layer 16. The acquisition layer 18 also may be substantially larger or smaller than the storage layer 16. In the embodiment depicted in FIG. 2, the acquisition layer 16 is substantially rectangular in shape, however, it may also have rounded ends or other shapes, such as an “I” shape or a “T” shape.

The acquisition layer 18 preferably has a front acquisition layer edge 58 defining the forward limit of the acquisition layer 18, and a rear acquisition layer edge 60 defining the rearward limit of the acquisition layer 16. A central acquisition layer region 62 may be located between the front and rear acquisition layer edges 58, 60.

The acquisition layer 18 preferably is made, at least partly, from a foam material having substantially no capillary pore orientation, although it may be comprised at least partly of any foam material so long as it retains the desirable features described herein. In one embodiment of the invention, the foam has the ability to absorb fluid striking the garment rapidly, hold the fluid with minimal fluid movement due to gravity, and release the fluid to the storage layer 16.

In one embodiment, the pore size of the foam material is selected to be large enough to accommodate fluids striking the garment 10, but small enough to hold the fluid against gravity long enough to allow the storage layer 16 to absorb the retained fluids. In another embodiment of the invention, the hydrophilicity of the foam material is adjusted or modified in all or part of the acquisition layer 18 in order to improve the performance of the acquisition layer 18. The foam may be selected from a number of materials, including, but not limited to, melamineformaldehyde-based foam materials, and polyurethane foams. The invention preferably, although not necessarily, does not include foams made using a high internal phase emulsion process (e.g., HIPE foams).

A preferred acquisition layer 18 for use in the invention includes at least one foam material, whereby the fluid drain rate is such that the acquisition layer 18 loses less than about 55% of its fluid after 60 seconds when subjected to the fluid drain rate test outlined in the Test Methods section below. Preferably, the acquisition layer loses less than about 53%, and more preferably, less than about 50% of its fluid after 60 seconds.

Typical fluid acquisition layers that are made of, for example, carded thermal bonded materials having a basis weight on the order of about 40 grams/m², are designed to acquire and distribute fluids quickly. Thus, these conventional acquisition layers will lose fluid much more rapidly than the acquisition layers of the present invention that are designed to at least partially resist the flow of liquid due to gravity.

Any material can be used to manufacture the fluid acquisition layer 18, so long as it provides the fluid drain rate and/or absorbent capacity under load and/or peak capacity values described herein. It is particularly preferred in the invention that the fluid acquisition layer 18 be comprised of at least one foam material, which preferably comprises a polymer. Any polymeric foam material effective in conferring to the absorbent article the fluid drain rate and/or absorbent capacity characteristics recited herein are suitable. Persons of skill in the art would readily be able to select and utilize such polymers to implement the present invention, based upon the guidance provided herein.

Non-limiting exemplary polymers suitable in implementation of the present invention include polymers selected from the group consisting of polyurethanes, polyethylenes, polypropylenes, polyacrylics, polyamides, polyvinyl chlorides, epoxys, polystyrenes, melamine-formaldehyde polymers and combinations thereof. The foam-containing fluid acquisition layer 18 preferably comprises any suitable non-yellowing polymeric foam material, and most preferably is comprised of at least an aliphatic isocyanate-derived polyurethane foam. Other polymeric foams may be used together with the aliphatic isocyanate-derived polyurethane foam so long as the absorbent article maintains its desirable strikethrough and rewet properties.

Aliphatic isocyanate-derived polyurethane foams generally are known in the art. Skilled artisans are capable of making a suitable non-yellowing polyurethane foam using the guidelines provided herein. Given the desirable properties of the foam, a number of polymeric foam materials may be made in a variety of thicknesses. These materials then can be constructed into a fluid acquisition layer using the guidelines provided herein, and tested in accordance with the procedures outlined herein. Those polymeric foams, when constructed into an absorbent article and tested to have a fluid drain rate such that it loses less than 55% of its fluid after 60 seconds, and/or when tested together with the remaining portions of the absorbent garment to have an absorbent capacity under load in the front portion thereof of greater than about 1.1 g/m², and an absorbent capacity under load in the rear portion thereof within the range of from about 0.5 to about 0.9 g/m², are useful polymeric foams in the present invention. These materials also preferably will have a peak capacity of greater than about 25 grams of fluid, when measured in accordance with the fluid drain rate test described in detail in the Testing Methods section below. Preferably, the peak capacity is greater than about 30 grams, more preferably greater than about 35 grams, and most preferably greater than about 40 grams.

While not preferred in the present invention, it is possible to modify existing foam materials to be more hygienic and/or non-yellowing, and then use these foam materials in the invention, so long as the foams have the desirable characteristics described above. For example, a conventional melamine-formaldehyde foam material available from BASF Corp., Research Triangle Park, N.C., designated BASOTECT®, has a formaldehyde content that typically is too high for use in a baby diaper. Such foams can be modified, however, to reduce the residual formaldehyde content, by, for example, using less formaldehyde or including a formaldehyde scavenger during production of the foam to thereby provide a foam more suitable for use in a baby diaper. Those skilled in the art are capable of modifying conventional melamine-formaldehyde foam materials to reduce the residual formaldehyde content, and then test the foam materials in accordance with the present invention to determine suitable foams for use herein.

It is preferred that the polymeric foam materials be comprised of an aliphatic isocyanate-derived polyurethane foam material, although such a foam material is not necessary for the invention. Aliphatic isocyanates are preferred starting materials due to the environmental hazards associated with using toluene or benzene-based isocyanates. In addition, aliphatic isocyanate-derived polyurethane foams do not yellow like other polyurethane foams, thereby making their use in a wearable absorbent article more desirable. Aromatic isocyanates can be used, however, to the extent the user is not concerned with yellowing or other disadvantages that may or may not result from their use.

Suitable aliphatic isocyanate-derived polyurethane foam materials can be made by using a prepolymerization process, or by directly polymerizing an aliphatic isocyanate with a polyol, in the presence of a catalyst and optionally water. It generally is preferred to form a prepolymer adduct having terminal isocyanate groups.

Suitable aliphatic polyisocyanates used to prepare the isocyanate-terminated prepolymer that may be employed in this invention may be either a compound composed only of an aliphatic chain or an alicyclic compound or a compound with an aromatic ring present in an aliphatic chain. Concrete examples thereof are hexamethylene diisocyanate, hexamethylene triisocyanate, bicycloheptane triisocyanate, undecanetriisocyanate, lysine ester triisocyanate, isophorone diisocyanate, dicyclohexylmethane diisocyanate, methylcyclohexane diisocyanate, dimethylcyclohexane diisocyanate, xylylene diisocyanate, tetramethylxylylene diisocyanate, their dimers, and their trimers. Of these, hexamethylene diisocyanate is especially preferable.

In accordance with the present invention, the aliphatic polyisocyanate component generally is in the form of an NCO prepolymer or a polyisocyanate adduct, more preferably a polyisocyanate adduct. Suitable polyisocyanate adducts for the present invention may be based, for example, on organic aliphatic diisocyanates including, for example, 1,4-tetramethylene diisocyanate, 1,6-hexamethylene diisocyanate, 2,2,4-trimethyl-1,6-hexamethylene diisocyanate, 1,12-dodecamethylene diisocyanate, cyclohexane-1,3- and -1,4-diisocyanate, 1-isocyanato-2-isocyanatomethyl cyclopentane, 1-isocyanato-3-isocyanatomethyl-3,5,5-trimethyl-cyclohexane (isophorone diisocyanate or IPDI), bis-(4-isocyanatocyclohexyl)methane, 2,4′-dicyclohexylmethane diisocyanate, 1,3- and 1,4-bis-(isocyanatomethyl)-cyclohexane, bis-(4-isocyanato-3-methylcyclohexyl)methane, α,α,α′,α′-tetramethyl-1,3- and/or -1,4-xylylene diisocyanate, 1-isocyanato-1-methyl-4(3)-isocyanatomethyl cyclohexane, 2,4- and/or, 6-hexahydrotoluylene diisocyanate, and mixtures thereof. It is preferred that the isocyanate be based on 1,6-hexamethylene diisocyanate. These and other suitable aliphatic isocyanates are described in, for example, U.S. Pat. Nos., 5,147,897, 5,164,421, 5,502,147, and WO 01/55242, the disclosures of which are incorporated by reference herein in their entirety.

Suitable polyisocyanate adducts containing biuret groups include polyisocyanates such as those described, for example, in U.S. Pat. Nos. 3,124,605, 3,358,010, 3,644,490, 3,862,973, 3,906,126, 3,903,127, 4,051,165, 4,147,714, and 4,220,749, the disclosures of which are herein incorporated by reference in their entirety. As set forth in these patents, these biuret group-containing polyisocyanates may be prepared by using co-reactants such as water, tertiary alcohols, primary and secondary monoamines, and primary and/or secondary diamines. These polyisocyanates preferably have an NCO content of 18 to 22% by weight and an average NCO functionality of 2.3 to 4.0, preferably of 3 to 3.5.

Suitable polyisocyanates containing isocyanurate groups include compounds such as those described, for example, in U.S. Pat. Nos. 4,288,586 and 4,324,879, the disclosures of which are herein incorporated by reference in their entirety; European Patents 3,765, 10,589 and 47,452, the disclosures of which are herein incorporated by reference; and German Offenlegungsschrifien 2,616,416, herein incorporated by reference. The isocyanato-isocyanurates generally have an average NCO functionality of 2.3 to 4.0, preferably of 3 to 3.5, and an NCO content of 5 to 30%, preferably 10 to 25% and most preferably 15 to 25% by weight.

Uretdione diisocyanates may be prepared by oligomerizing a portion of the isocyanate groups of a diisocyanate in the presence of a trialkyl phosphine catalyst, and may be used in admixture with other aliphatic and/or cycloaliphatic polyisocyanates, particularly the isocyanurate group-containing polyisocyanates described hereinabove.

Urethane group-containing polyisocyanates which may be prepared in accordance with the process disclosed in U.S. Pat. No. 3,183,112, herein incorporated by reference in its entirety, by reacting excess quantities of polyisocyanates, preferably diisocyanates, with low molecular weight glycols and polyols having molecular weights of less than 400, such as trimethylol propane, glycerine, 1,2-dihydroxy propane and mixtures thereof, also are useful in the present invention.

Allophanate group-containing polyisocyanates include, for example, those prepared according to the processes disclosed in U.S. Pat. Nos. 3,769,318, 4,160,080 and 4,177,342, the disclosures of which are herein incorporated by reference. Isocyanurate and allophanate group-containing polyisocyanates include, for example, those which may be prepared in accordance with the processes set forth in U.S. Pat. Nos. 5,124,427, 5,208,334 and 5,235,018; the disclosures of which are herein incorporated by reference. These polyisocyanates containing isocyanurate and allophanate groups preferably have an NCO content of 16 to 22% by weight, most preferably of 18 to 21% by weight.

Suitable carbodiimide group-containing and uretone imine group-containing polyisocyanates for use in the present invention include, for example, those that may be prepared by oligomerizing di- or polyisocyanates in the presence of known carbodiimidization catalysts such as described in, for example, German Patentschrifien 1,092,007, herein incorporated by reference, U.S. Pat. No. 3,152,162, herein incorporated by reference, and German Offenlegungschrifien 2,504,400, 2,537,685 and 2,552,350, the disclosures of which are herein incorporated by reference.

The aforementioned isocyanates preferably are reacted with a polyol to prepare the polyurethane foam material in the present invention. Any suitable polyol can be used so long as the ultimate foam produces an absorbent article having the properties described herein. A suitable polyol employed to form a prepolymer using the aliphatic polyisocyanate, is a polyol having a number average molecular weight of 100 to 5,000, preferably 200 to 3,000, and containing on the average 2 to 3 functional groups. Examples of the polyol useful in this invention include polyether polyols such as adducts [e.g., polyethylene oxide, polypropylene oxide, and poly(ethylene oxide-propylene oxide) copolymer] of dihydric or trihydric alcohols (e.g., ethylene glycol, propylene glycol, glycerol, hexanetriol, and triethanolamine) and alkylene oxides (e.g., ethylene oxide, propylene oxide, and butylene oxide), and polytetramethylene ether glycol obtained by subjecting tetrahydrofuran to ring opening polymerization; lactone-type polyester polyols obtained by adding lactones such as caprolactone, glycolide and lactide to the above dihydric or trihydric alcohols via ring opening; compounds obtained by condensing the above dihydric or trihydric alcohols with hydroxycarbonic acids such as glycolic acid, lactic acid, and salicylic acid; compounds obtained by condensing dicarboxylic acids such as oxalic acid, maric acid, succinic acid, glutaric acid, phthalic acid, and adipic acid with diols such as ethylene glycol, and propylene glycol; and condensed polyester polyols obtained by adding acid anhydrides such as phthalic anhydride with diols. The prepolymer can be prepared from the aliphatic polyisocyanate and the polyol in a known manner by the addition reaction of the respective components. It is preferred to use a reaction ratio of the polyisocyanate and the polyol, (an NCO/OH ratio) of about 1.4 to 2.6, preferably from about 1.5 to 2.5.

A prepolymer having the isocyanate groups in substantially all the molecule terminals can be made from the above described reactions. The prepolymer then preferably is reacted with water in the presence of the hardening catalyst. The hardening reaction proceeds by chain extension accompanying a urea bond that occurs by reacting an amino group resulting from the reaction of the terminal isocyanate group of the prepolymer and water with the terminal isocyanate group of the other prepolymer.

The amount of water used in the reaction can be anywhere from about 0.4 to 5 times, preferably 0.5 to 4.5 times the isocyanate equivalent of the prepolymer. In accordance with one aspect of this invention in which the polyurethane urea foam is formed by the reaction of the isocyanate-terminated prepolymer and water, it has been found that the reaction can of course be carried out by using a highly active hardening catalyst such as amines or organometallic compounds. Even when one employs carboxylic acid metal salts of low toxicity, the hardening reaction rapidly proceeds, and a less toxic non-yellowing polyurethane foam can be prepared. Those skilled in the art will appreciate that the same hardening reaction can take place without forming the aforementioned prepolymer, and skilled artisans are capable of making a suitable foam for use in the present invention using the guidelines provided herein.

Examples of suitable carboxylic acid metal salts useful as an active hardening catalyst include alkali metal salts, lead salts, alkaline earth metal salts, especially calcium salts, of aliphatic carboxylic acids which are C₂-C₁₀alkane acids such as acetic acid, propionic acid, butyric acid, valeic acid, caproic acid, caprylic acid, capric acid, and 2-ethylhexanoic acid. Of these, the calcium or sodium salts are preferable. The amount of the carboxylic acid metal salt can be 0.1 to 5 parts by weight, preferably 1 to 3 parts by weight per 100 parts by weight of the prepolymer.

The polyurethane foam useful in the invention also may be formed by the reaction of the isocyanate-terminated prepolymer and water in the presence of an amine-type catalyst. The amine-type catalyst can be an amine-type catalyst well known in the urethane field; a tertiary amine is especially preferable. Examples of suitable tertiary amine include monoamines such as triethylamine, and dimethyl cyclohexylamine; diamines such as tetramethylethylenediamine, and tetramethylhexanediamine; triamines such as tetramethylguanidine; cyclic amines such as triethylenediamine, dimethylpiperadine, and methylmorphorine; alcoholamines such as dimethylaminoethanol, trimethylaminoethylethanolamine, and hydroxyethylmorphorine; ether amines such as bisdimethylaminoethyl ethanol; diazabicycloalkenes such as 1,5-diazabicyclo(5,4,0)undecene-7 (DBU), and 1,5-diazabicyclo(4,3,0)nonene-5; and organic acid salts of the diazabicycloalkenes such as phenol salt, 2-ethylhexanoate and formate of DBU. Of these, a diazabicycloalkene such as that disclosed in U.S. Pat. No. 5,147,897, and a salt of it and an organic acid are especially preferable. These amines can be used either singly or in combination.

The amine-type catalyst can be used in an amount of usually 0.1 to 10 parts by weight, more preferably 0.4 to 4 parts by weight.

It is possible in the invention, that two or more prepolymers different in isocyanate portion and/or polyol portion may be mixed and used, or the aforesaid polyol having the average molecular weight of 100 to 5,000 and containing on the average 2 to 3 functional groups may be added to the hardening reaction system, as required. This enables modification of a viscosity of an expansion starting solution composed of a prepolymer, water, a catalyst, etc., increase in compatibility thereof and control of properties of the hardened product. Even in the method of this invention that conducts the chain extension chiefly by the formation of the urea bond via the reaction of the isocyanate group and water, the polyol can be incorporated into the hardened product by the formation of the urethane bond via the reaction of the hydroxyl group and the isocyanate group. The amount of the polyol can be usually 60% or less, preferably 50% or less.

A particularly preferred method of making an aliphatic isocyanate-derived polyurethane foam for use in the invention is described in WO 01/55242, the disclosure of which is incorporated by reference herein in its entirety. A flexible, semi rigid or rigid, preferably open-celled polyurethane foam can be made using at least one complexing agent selected from ethylenimine, polyethylenimine, polyvinylamine, carboxy-methylated polyethylenimines, phosphono-methylated polyethylenimines, quaternized polyethylenimines and/or dithiocarbamitized polyethylenimines.

Examples of suitable complexing agents are: ethylenimine, polyethylenimines having a molecular weight range from 500 to 30,000 g/mol, carboxy-methylated polyethylenimines having a molecular weight range from 1000 to 50,000 g/mol, phosphono-methylated polyethylenimines having a molecular weight range from 1000 to 50,000 g/mol, quaternized polyethylenimines having a molecular weight range from 1000 to 50,000 g/mol, dithiocarbamitized polyethylenimines having a molecular weight range from 1000 to 50,000 g/mol, and polyvinylamines.

These particularly preferred complexing agents can be applied to the polyurethane foam by two different methods. Firstly, the production of the polyurethane foam by reaction of polyisocyanates with compounds having at least two hydrogen atoms that are reactive toward isocyanates can be carried out in the presence of the complexing agent. However, the complexing agents also can be reacted with isocyanate to form prepolymers, i.e. reaction products of the complexing agents and polyisocyanates that have free isocyanate groups at the end of the chain. Prepolymers and pseudoprepolymers and their preparation generally are known and have been described above.

A second method is to impregnate the polyurethane foam with the complexing agent after production of the foam. Treatment of the foam with a liquid complexing agent or a solution of the solid or liquid complexing agent in a suitable solvent is believed to result in the foam being impregnated with the complexing agent. Suitable solvents include protic solvents, for example water, acetone, i-propanol or methyl ethyl ketone, or haloalkanes such as 1,2-dichloromethane. The solvent can subsequently be removed from the foam impregnated with the complexing agent. This can be achieved by simple application of a vacuum or by drying at up to 50° C. Thermal treatment at from 50 to 150° C. for from 4 to 72 hours enables the complexing agents to react with the foam and thus be covalently bound thereto.

To achieve better immobilization of the complexing agent, the foam can be produced using an excess of isocyanate. In this case, the complexing agent can be fixed to the foam framework via remaining isocyanate groups.

The foam also can be impregnated beforehand with a dilute isocyanate solution. A foam prepared in this manner then may be impregnated with the solution of complexing agent. Here too, the complexing agent can be bound to the foam via the isocyanate groups.

In a post-impregnation of the foams with a solution of complexing agent, the absorption capacity of the foam also is dependent on the type and polarity of the solvent in which the active compound has been dissolved. Using acetone as the preferred solvent for the complexing agent increases the capacity of the foam for the complexing agent.

The complexing agents applied to the foam by impregnation can, if desired, be crosslinked on the foam in a further step. Examples of suitable crosslinkers include nonvolatile PEC bisglycidyl ethers or comparable functional compounds, or polycarboxylic acids. The temperatures required for crosslinking are typically around 80° C. for most cross-linking, and from about 120 to about 130° C. for the polycarboxylic acids.

The polyurethane foams that are particularly useful in the present invention contain anywhere from about 0.1 to about 50% by weight of the aforementioned complexing agent, based on the weight of the foam.

It is advantageous to make the polyurethane foams hydrophilic, as a result of which the foam can be wetted with a liquid, such as urine, and the like. The hydrophilicity of the polyurethane foams can be increased, for example, by use of polyetherols having a high ethylene oxide content in the chain.

The production of polyurethane foams by reacting isocyanates, for example polyisocyanates, with compounds having at least two hydrogen atoms that are reactive toward isocyanates generally is known. To produce the polyurethanes of the present invention, the isocyanates can be reacted with the compounds having at least two active hydrogen atoms in the presence of blowing agents and, if desired, catalysts and/or auxiliaries and/or additives. Here, the compounds having at least two hydrogen atoms that are reactive toward isocyanate groups and the above mentioned blowing agents, catalysts and auxiliaries and/or additives frequently are combined to form a polyol component before the reaction, and this polyol component then is brought into contact with the isocyanate component.

The particularly preferred starting materials that are possible for carrying out the process of the present invention, i.e. the isocyanates, the compounds having at least two active hydrogen atoms, the blowing agents and, if desired, the catalysts and/or the auxiliaries and/or additives, are as follows:

The isocyanates preferably are polyisocyanates, particularly preferably diisocyanates, and most preferably organic diisocyanates, although it is possible to use the customary and known (cyclo)aliphatic and aromatic polyisocyanates. Examples of aromatic polyisocyanates are toluylene 2,4 and 2,6-diisocyanate (TDI), diphenylmethane 4,4′-, 2,4′- and 2,2′diisocyanate (MDI), polyphenylene-polymethylene polyicocyanatos (crude MDI), and naphthylene 1.5-diisocyanate.

Examples of (cyclo)aliphatic diisocyanates or triisocyanates are tetramethylene 1,4-diisocyanate, hexamethylene 1,6-diisocyanate, isophorone diisocyanate, 2 methylpentamethylene diisocyanate, 2,2,4- or 2,4,4-trimethylhexamethylene 1,6-diisocyanate, 2-butyl-2-ethylpentamethylene diisocyanate, 1,4-diisocyanatocyclohexane, 3-isocyanatomethyl-1-methyl 1-isocyanatocyclohexane, isocyanatopropylcyclohexyl isocyanate, xylylene diisocyanate, tetramethylxylylene diisocyanate, bis(4-isocyanatocyclohexyl)methane, lysine ester isocyanates, 1,3- or 1,4-bis(isocyanatomethyl)cyclohexane, 4-isocyanatomethyloctamethylene 1,8 diicocyanate and mixtures thereof or the oligoisocyanates or polyisocyanates prepared there from.

While the aforementioned aromatic and cycloaliphatic isocyanates may be used to make suitable foams, it is preferred not to use them for use in an absorbent garment to be used by an infant, since consumers typically perceive their use to be hazardous, regardless of whether the final product is hazardous or not. It is particularly preferred in the invention to use the aliphatic isocyanates selected from tetramethylene 1,4-diisocyanate, hexamethylene 1,6-diisocyanate, isophorone diisocyanate, 2 methylpentamethylene diisocyanate, 2,2,4- or 2,4,4-trimethylhexamethylene 1,6-diisocyanate, 2-butyl-2-ethylpentamethylene diisocyanate, and mixtures thereof or the oligoisocyanates or polyisocyanates prepared there from

The oligoisocyanates or polyisocyanates can be prepared from the above-mentioned diisocyanates or triisocyanates or mixtures thereof by coupling by means of urethane, allophanate, urea, biuret, uretdione, amida, isocyanurate, carbodiimide, uretonimine, oxadiazinetrione or iminooxadiazinedione structures.

The isocyanates mentioned above also can be modified, for example by incorporation of carbodiimide groups. The polyisocyanates also frequently are used in the form of prepolymers. These prepolymers are reaction products of the polyisocyanates mentioned above with polyol components. It is preferred to use isocyanate prepolymers, i.e. reaction products of polyols and polyisocyanates that have free isocyanate groups at the end of the chain. The prepolymers and pseudoprepolymers and wtheir preparation generally are known and have been described in the literature. In the process of the present invention, it is particularly preferred to use prepolymers having an NCO content in the range from about 3.5 to about 25% by weight.

In another preferred embodiment of the process of the present invention, biuretic, isocyanurates and allophanates based on aliphatic isocyanates are used as the isocyanate component.

Any compound having at least two active hydrogen atoms can be used in the invention. Preferred compounds include polyester alcohols and particularly preferred are polyetherols having a functionality of from 2 to 8, in particular from 2 to 4, preferably from 2 to 3, and a molecular weight in the range from 1000 to 8500 g/mol, preferably from 1000 to 6000. The compounds having at least two active hydrogen atoms also include chain extenders and cross linkers that may be additionally used, if desired. Chain extenders and cross linkers preferably include 2- and 3-functional alcohols having molecular weights of less than 1000 g/mol, in particular in the range from 60 to 150. Examples include ethylene glycol, propylene glycol, diethylene glycol, dipropylene glycol, polyethylene glycol having a molecular weight of less than 1000, polypropylene glycol having a molecular weight of less than 1000 and/or 1,4 butanediol. Diamines also can be used as cross linkers. If chain extenders and cross linkers are used, their amount preferably is up to 5% by weight, based on the weight of the isocyanates.

It is possible in the invention to use any of the known and customary polyurethane formation catalysts as catalysts for producing the polyurethane foams of the present invention. For example organic tin compounds such as tin diacetate, tin dioctoate, dialkyltin dilaurate, and/or strongly basic amines such as triethylamine, pentamethyldiethylenetriamine, bis(dimethylaminoethyl)ether, 1,2-dimethylimidazole, dimethylcyclohexylamine, dimethylbenzylamine or preferably triethylenediamine are used. The catalysts preferably are used in an amount of from 0.01 to 5% by weight, preferably from 0.05 to 2% by weight, based on the weight of the isocyanates.

It is preferred to use water as a blowing agent for producing the polyurethane foams, because the water reacts with the isocyanate groups to liberate carbon dioxide. Together with or in place of water, it also is possible to use physically acting blowing agents, for example hydrocarbons such as n-pentane, isopentane or cyclopentane or halogenated hydrocarbons such as tetrafluoroethane, pentafluoropropane, heptafluoropropane, pentafluorobutane, hexafluorobutane or dichloromonofluoroethane, or acetals such as methylal. The amount of physical blowing agent preferably is within the range of from about 1 to about 15% by weight, in particular from 1 to 10% by weight, and the amount of water preferably is within the range from 0.5 to 10% by weight, in particular from 1 to 5% by weight, based on the weight of the compounds having at least two active hydrogen atoms.

In the production of the polyurethane foams of the present invention, the polyisocyanates and the compounds having at least two hydrogen atoms that are reactive toward isocyanate groups preferably are reacted in such amounts that the equivalence ratio of isocyanate groups to the sum of the active hydrogen atoms is 0.7-1.8:1, preferably 0.7-1.20:1.

The polyurethane foams preferably are produced by the one-shot method, for example with the aid of the high-pressure or low-pressure technique. The foams can be produced in open or closed metallic molds or by continuous application of the reaction mixture to conveyor belts for producing foam blocks.

It is particularly advantageous to employ the two-component process in which, as indicated above, a polyol component and an isocyanate component are prepared and foamed with one another. The components preferably are mixed at from 15 to 90° C., preferably from 20 to 60° C. and particularly preferably from 20 to 35° C., and introduced into a mold or applied to a conveyor belt. The temperature in the mold usually is in the range from 20 to 110° C., preferably from 30 to 60° C. and particularly preferably from 35 to 55° C.

In the direct addition of the complexing agent during the production of the polyurethane foams, the complexing agent can be added to either the polyol component or the isocyanate component. Preference is given to adding the complexing agent to the polyol component.

In the method of this invention, additives such as surface active substances, foam stabilizers, cell regulators, blowing agents, fire retardants, chain extenders, crosslinking agents, external and internal mold release agents, fillers, pigments, hydrolysis inhibitors, and fungistatic and bacteriostatic substances may be added as is often the case with production of ordinary polyurethanes. Moreover, in order to react the remaining isocyanate groups left after expansion, the foam may be heated to conduct post-hardening.

Particularly preferred components used to prepare the foams of the present invention are the aliphatic isocyanates and oligomers thereof, MDI available under the tradename LUPRANAT®, BASF Aktiengesellschaft, Ludwigshafen, Germany, polyetherols available under the tradename LUPRANOL®, BASF Aktiengesellschaft, Ludwigshafen, Germany, catalysts such as bis(dimethylaminoethyl)ether, available under the tradename LUPRAGEN®, BASF Aktiengesellschaft, Ludwigshafen, Germany, polyethylenimines as complexing agents, available under the tradename LUPASOL®, BASF Aktiengesellschaft, Ludwigshafen, Germany, aminopropylimindazol, tetramethylhexamethylene diamine, as well as suitable surfactants, stabilizers and other conventional additives.

The acquisition layer 18 may be made entirely from a foam material, such as those described above, or the acquisition layer 18 may be made only partly from such a material. In one embodiment of the present invention, the acquisition layer 18 is comprised of several zones having physical properties that differ from zone to zone. In such an embodiment, at least one of the zones is comprised of a foam material. Such an embodiment may be used to provide tailored properties to different regions of the garment. For example, it may be desired to provide a different material in the region stricken primarily by urine than the in the region that primarily handles solid fecal matter.

FIG. 6 depicts an acquisition layer 18 in the fully flattened position. In the embodiment depicted in FIG. 6, there are three zones A, B, C, each of which extends across the entire width of the acquisition layer 18. The first zone A extends rearward from the front acquisition layer edge 58, the second zone B is in the central acquisition layer region 62, and the third zone C extends between the second zone B and the rear acquisition layer edge 60. The first zone A may be comprised of a non-foam material that is selected to provide a longitudinal wicking force to the fluids, thereby drawing the fluid forward where it may be distributed into the foremost region of the storage layer 16. The second zone B may be comprised of a foam material that provides little or no wicking or capillary action, and that holds fluid against gravity until it is absorbed by the adjacent region(s) of the storage layer 16. The third zone C may be comprised of a non-foam material that is selected to provide a longitudinal wicking force to the fluids, thereby drawing the fluid backward, where it may be distributed into the rearmost region of the storage layer 16.

Furthermore, in the embodiment depicted in FIG. 6 the zones A, B, C may have different hydrophilicities, such that the first zone A is more hydrophilic than the second zone B, and the second zone B is more hydrophilic than the third zone C. In such an embodiment, fluids would tend to be drawn from the zone having a lower hydrophilicity into the zone having a higher hydrophilicity. Such an arrangement would further resist downward fluid flow caused by gravity, and would help distribute fluid to the foremost region of the storage layer. Other combinations of zone hydrophilicity and material compositions also may be used to promote fluid flow into the front-most and/or rear-most portions of the garment 10, in order to optimally distribute fluid during nighttime use of the garment 10.

In an embodiment with an acquisition layer 18 having different zones, one zone may be located entirely within the front half 42, and may be located proximal to the “strike region,” or “insult point” where urine is most likely to strike the garment 10.

Still referring to FIG. 6, in one embodiment of the invention, the acquisition layer 18 may be thicker in some areas than others. In an embodiment in which the acquisition layer 18 is comprised of different zones, the variations in the thickness may correspond with the zones A, B, C, or they may be independent of the zones A, B, C. In one embodiment, the second zone B is comprised of a foam material and is located in the strike region. In this embodiment, the second zone thickness T_{B} may be greater than the first and third zone thicknesses T_{A}, T_{C} or the second zone (B) absorbent capacity may be greater than the absorbent capacity of the other zones A and C. Such an embodiment may be employed to provide greater fluid surge handling capacity in the strike region of the garment 10, without making the garment 10 unnecessarily thick in the other zones A, C, and therefore less comfortable. Other benefits may be obtained by this and other embodiments of the invention in which the absorbent capacity of the acquisition layer 18 varies.

In one embodiment of the invention, the acquisition layer 18 and the storage layer 16 work together to provide substantial fluid absorbing capacity in the front half 42 of the garment 10 while the wearer is positioned in an upright standing or sitting position. In such an embodiment, the acquisition layer absorbs fluid in the front half 42 of the garment and retains the fluid, against the pull of gravity, long enough for the fluid to be absorbed by the portion of the storage layer 16 located in the front half 42 of the garment 10.

FIG. 7 is a cut away side view of an embodiment of the present invention shown in the upright position, and indicating the flow of liquid within the garment 10 with arrows. The entering fluid, strikes the garment 10 at the strike region 64, or “insult point,” and passes through the fluid pervious topsheet 14. Fluid enters the second zone B of the acquisition layer 18, which is located in the strike region 64. The second zone B, which preferably is a foam material, tends to distribute the fluid equally in all directions within the second zone B, and holds the fluid against the force of gravity G long enough to be absorbed by the adjacent region of the storage layer 16.

Some of the fluid entering the acquisition layer 18 in the second zone B is redistributed towards the top edge 66 of the second zone. Once the fluid reaches the first zone A of the acquisition layer 18, which preferably is comprised of a material having capillary channels oriented vertically, the fluid is drawn upwards against gravity G by capillary action until it is absorbed by the storage layer 16.

The remaining fluid entering the second zone B migrates towards the lower edge 68 of the second zone B until it contacts the third zone C of the acquisition layer 18. The third zone C also preferably is comprised of a material having capillary channels oriented vertically, and the fluid contacting it is drawn by capillary and gravity G towards the crotch region 22 of the garment 10, where it is absorbed by the adjacent region of the storage layer 16.

In the embodiment depicted in FIG. 7, the foam-containing second zone B of the acquisition layer 18 holds fluid longer than conventional carded thermal bonded layers would, (typically having a basis weight of about 40 g/m²), allowing more of the fluid to be absorbed by the adjacent region of the storage layer 16. Thus, absorbent material can be removed from the crotch 22 and added to the front waist region 20 (42) of the garment 10. Such a garment would provide adequate daytime (i.e., upright) fluid handling and storage performance. Such a garment also would provide improved nighttime performance because more of the storage layer's 16 absorbent capacity is placed in the front waist region 20 (42), where the fluid tends to settle when the garment 10 is used in a horizontal position. The garment 10 also is suitable for night time use because the acquisition layer 18 can hold the fluid against the force of gravity to thereby distribute it more efficiently to more suitable areas of storage layer 16.

The physical characteristics of the absorbent article of the invention (e.g., fluid drain rate and absorbent capacity under load) can be affected by a variety of physical properties of the foam used in accordance with an implementation of the present invention. These properties include the thickness, the density, the basis weight, and the change in basis weight of the foam material. The change in basis weight of the foam materials is, in essence, a measure of the solubles content of the foam. The more solubles present in the foam material, the greater the change in basis weight. These factors also influence the cost effectiveness of the absorbent articles.

The thickness of the foam material is in essence the thickness of the fluid acquisition layer 18, and can be measured using any mechanism capable of measuring a thickness. The basis weight of the foam material is its dry weight divided by the area of the material weighed, and typically is represented in grams/m². The density and change in basis weight are determined in accordance with the Testing Methods described in more detail below.

Particularly preferred foam materials for use in the fluid acquisition layer 18 of the invention have a thickness within the range of from about 2.95 to about 7.0 mm, more preferably from about 3.25 to about 5.5 mm, and most preferably from about 3.5 to about 5.0 mm. The density of the preferred foam materials for use in the fluid acquisition layer 18 of the invention preferably is less than about 0.080 g/cm³, more preferably is less than about 0.074 g/cm³, and most preferably is less than about 0.073 g/cm³.

The basis weight of preferred foams for use in the fluid acquisition layer 18 of the invention can range anywhere from about 200 to about 500 g/m², preferably from about 210 to about 400 g/m², and most preferably from about 225 to about 350 g/m². It also is preferred to use foam materials having a change in basis weight (absolute value, since the change typically is a negative value) of less than about 30 g/m², more preferably less than about 20 g/m², and most preferably less than about 17 g/m².

The invention now will be explained by reference to the following non-limiting examples.

### EXAMPLES

Sample Preparation:

Absorbent articles were prepared for testing in accordance with the following procedure. Conventional absorbent articles were constructed using known apparatus and known materials, including a topsheet 14, back sheet 12, storage layer, or absorbent core, 16 and fluid acquisition layer 18. The conventional fluid acquisition layer used herein for comparison purposes is a 40 g/m² carded thermal bond material adhered to the topsheet 30. The conventional diapers were Stage 4, Ultras, available from Paragon Trade Brands, Norcross, Ga. The diapers comprised a core that was about 110 mm wide, 380 mm long, had a basis weight of about 1,200 to about 1,440 g/m² (without the foam), a density of 1.14 g/cm³, and the concentration of SAP in fluff pulp was about 40% by weight.

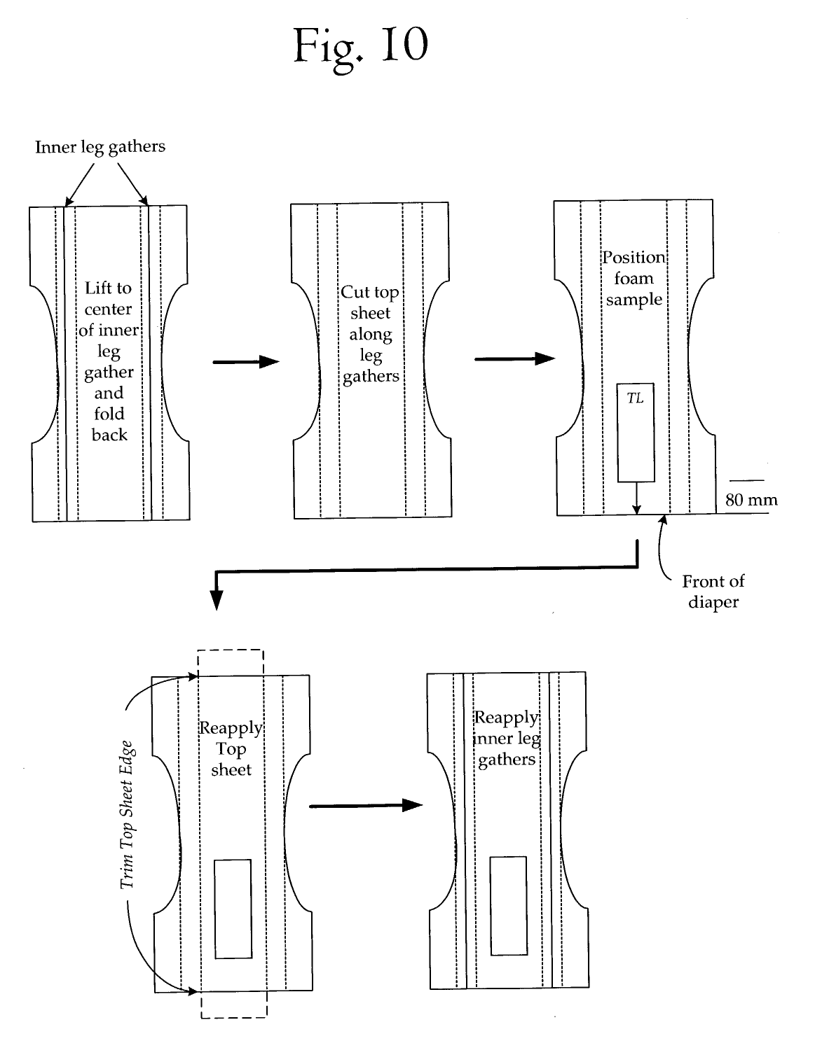

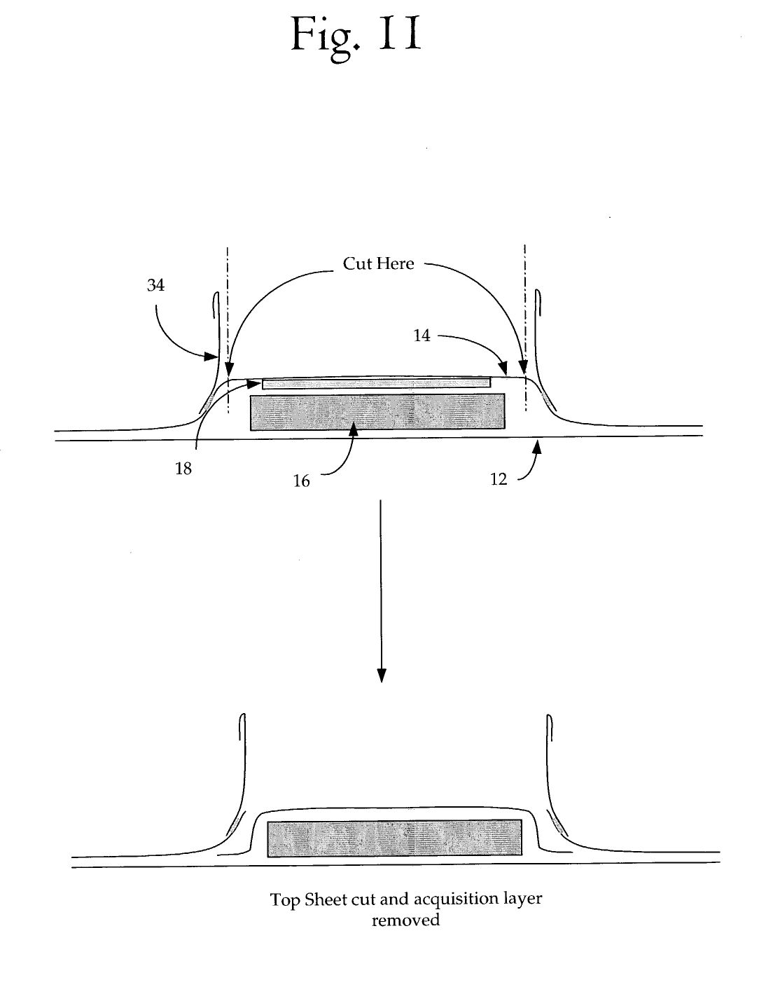

Referring now to FIGS. 10 and 11, the method of deconstructing the absorbent article, and then reconstructing it will be described. The conventional articles were de-constructed by removing the top sheet 14, and the fluid acquisition layer 18. The top sheet 14 was removed by first laying the garment flat, and clamping it to a stretch board. The inner leg gathers first are moved out of the way by folding them backwards, as shown in the upper left of FIG. 10. The folded portions of the inner leg gathers then can be clamped down, and the top sheet removed by cutting the top sheet along the leg gathers, as shown in the top center portion of FIG. 10. FIG. 11 provides a more detailed illustration of where the top sheet material is cut to enable its removal, along with the removal of the existing fluid acquisition layer 18, if present.

A foam material that was cut into an approximate 90 mm×165 mm rectangle was positioned on the garment, as shown in the upper right portion of FIG. 10. It is most preferable that the foam sample be placed about 80 mm from the front of the diaper, as indicated by the down arrow in the upper right graphic of FIG. 11. Another top sheet material, made from the same non-woven material as the top sheet 12 in the conventional article, was prepared separately and cut to the dimensions of the conventional absorbent article. The top sheet, which preferably was prepared having a spiral glue adhesive in an amount of about 4 g/m², then was reapplied and adhered to the remainder of the conventional article to reconstruct the absorbent article, as shown in the bottom left portion of FIG. 10. The leg gathers then can be reapplied to the top sheet by tape adhesive or other joining mechanisms to reconstruct the garment, as shown in the bottom right of FIG. 10.

The reconstructed absorbent article then was folded and placed in a compression cell with a weight placed on top for greater than about 16 hours, preferably 24 hours, before testing. The weight was large enough to completely cover a folded diaper, and exerts a pressure of about 1080 Pa (0.16 psi).

Other commercially available diapers were obtained, and were not modified as described above. The commercially available diapers were competitor brands in the same or similar size and shape category as the Stage 4, Ultras, available from Paragon Trade Brands, Norcross, Ga., used to hand reconstruct the diapers as described above.

Testing Procedures

Measuring Density

A foam sample first was prepared as follows. Foam samples of a predetermined size were cut from larger blocks of foam using a sharp reciprocating knife saw. Use of this or equivalent type of foam cutting device increased accuracy and specificity by serving to substantially eliminate edge flaws that may distort certain measurements made during the following test methods. Sample size specification also generally included a dimension for sample caliper or thickness. Caliper or thickness measurements for purposes of the present invention should be made when the foam sample is under a confining pressure of 350 Pa.

Density of the foam was determined using ASTM Method No. D3574-86. In particular, density measurements made according to the procedure were carried out on foam samples that had been preconditioned in a certain manner as specified in that test.

Density was determined by measuring both the dry mass of a given foam sample and its volume at 22±2° C. Volume determination on larger foam samples were calculated from measurements of the sample dimensions made under no confining pressure. Dimensions of smaller foam samples may be measured using a dial-type gauge using a pressure on the dial foot of 350 Pa (0.05 psi). Density was calculated as mass per unit volume. For purposes of this invention, density is hereinafter expressed in terms of grams per cubic centimeter (g/cc).

Measuring the Change in Basis Weight

The change in Basis Weight simply is the difference between the basis weight of the foam sample prior to, and after washing. Thus, soluble materials present in the foam material will be “washed out” and a higher solubles content will generate a higher change in basis weight. A foam sample prepared by cutting the foam into about a 2 inch diameter circle was conditioned for 24 hours at TAPPI conditions (70° F., 50% relative humidity), and then weighed to determine its dry basis weight. A 100 mm by 50 mm beaker then was filled with a fresh saline Triton X test solution (fresh solution used each time) prepared by the following procedure. Approximately 5-g of Triton X-100 were weighed and deposited into a clean, 200 ml flask. Then, about 18 g NaCl was weighed and transferred into the same 200 ml flask container with the Triton X-100, and diluted with de-ionized water to 200 ml liter. The solution then preferably was stirred. The test solutions were discarded if not used within seven days, or if the percent saline was not about 0.9% by weight, as measured using a refractometer.

The sample then was held about 2 inches above the surface of the solution, parallel to the upper surface, and dropped into the beaker. The timer was started once the sample contacted the solution. The timer was stopped once the sample wetted out and the top surface of the sample was substantially wet with solution. The samples then were retrieved from the solution, dried between paper towels, exposed to 32° C. heat for about 5 minutes in an oven, and then conditioned for 24 hours at TAPPI conditions (70° F., 50% relative humidity). The samples then were reweighed to calculate the final basis weight. The change in basis weight is the difference between the final basis weight and the initial dry basis weight. To the extent the foam materials contain soluble materials, the change in basis weight typically will be a negative number.

Measuring Absorbent Capacity Under Load

The absorbent capacity under load was measured in accordance with the following procedure, which is referred to herein as the PAD AUL test. At least six diapers were prepared and/or obtained as described in the Sample Preparation section above. The diapers were conditioned for at least 16 hours at TAPPI conditions (70° F., 50% relative humidity) prior to use. Samples then were obtained from the diapers by opening them up, marking the lateral centerline, and taking a front region sample at or about 4.5 cm from the centerline, and taking a back region sample at or about 3.0 cm from the opposite side of the centerline. Each sample was approximately 2 in. in diameter.

A 1% saline solution was prepared as follows. Approximately 50 g NaCL were weighed and add to a clean, dry 5,000 ml capacity flask or jug. The flask or jug then was filled to the 5,000 ml mark with deionized water.

The solution then was periodically stirred and the concentration of the solution was periodically measured with a refractometer and discarded if the percent saline was not 1%. The solution volume of a sample pan was calibrated by placing all six sample holders (holders to hold the front and rear region samples prepared above), and then slowly adding a measured amount of the 1% saline solution prepared above until the liquid volume rose to about ⅛ of an inch above the tops of the holes of every sample holder. This volume of saline solution was recorded as X, and the volume to add to a particular pan is defined as (X+120) ml (120 is 6 samples×20 ml for each holder). The sample holders were dried completely prior to the following testing protocol.

Each sample was weighed and the weight recorded. Each circular pad sample then was placed inside a respective sample holder with the top sheet side facing down, and the weight of the sample holder and the sample was recorded. A load of approximately 0.5 psi then was applied to the sample holder, x samples were added to the pan, and the pan was filled with (X+120) ml of the 1% saline solution at a temperature of about 73.4±2° F. All samples and sample holders and weights were placed in the solution at the same time. A timer then was started, and after ten minutes, the sample holders were removed in the same order in which they were placed in the pan, and allowed to drip for about 60 seconds. The 0.5 psi load then was removed, and the wet sample and sample holder were weighed.

The absorbent capacity under load for each of the samples was determined by subtracting the dry weight of the sample holder and the sample from the wet weight of the sample holder and sample. This value was recorded in grams. The absorbent capacity under load value in grams then was divided by the area of each sample, or by about 20.26 cm² (πr², where r is 1 inch) to obtain the value reported in g/cm².

Measuring the Fluid Drain Rate

A 0.9 wt % Triton X-100 saline solution was prepared as described above with respect to measuring the change in basis weight. A 1500 ml beaker was filled up to 10 mm from the top with the saline solution, and was placed on the bottom crosshead of an Instron apparatus, commercially available from Instron Corporation, Canton, Mass. The fluid acquisition layer material was placed in the top grips in the vertical position (longer dimension of the rectangular fluid acquisition layer, e.g., along horizontal axis 100 in FIG. 2), above the bottom crosshead. The beginning position of the Instron is set to a position where the fluid acquisition layer is not in contact with the saline solution in the beaker.

The load on the Instron then is balanced, and the initial weight set to zero. The Instron is programmed to move at about 304.8 mm/min to thereby submerge the fluid acquisition layer in the saline solution in the beaker. The Instron then stops so that the fluid acquisition layer is submerged as much as possible without the grips holding the acquisition layer touching the beaker of solution. After about 30 seconds of being submerged, the crosshead is programmed to move at about 1270 mm/min to the point where about 5 mm of the fluid acquisition layer still remains in the solution. The fluid acquisition layer now is draining.

During the fluid acquisition draining, the maximum load (peak load) is recorded, and the load at about 6 seconds after the crosshead stops. The maximum load is the peak capacity, and is represented herein in units of grams (g). The load is measured again at about 60 seconds after the crosshead stops, and this load is the value used to determine the percent fluid loss at 60 seconds. The percent fluid loss is the maximum load minus the load at 60 seconds, divided by the maximum load.

### Example 1

A conventional acquisition layer was obtained from a commercially available Stage 4, Ultras product available from Paragon Trade Brands, Norcross Ga., that is comprised of an about 36 g/m² carded thermal bonded material. This product was designated the “Control.” Another commercially available absorbent article sold by a competitor having a similar carded thermal bonded fluid acquisition layer also was tested. This product was designated H.

Conventional fluid acquisition layers comprised of carded, thermal bonded polyolefin materials also were modified such that the fibers were aligned either orthogonal to gravity, (designated BBA CD) or parallel to gravity (designated BBA MD). All of the conventional fluid acquisition layers had similar dimensions and properties when compared to the control, and consequently, only the physical properties of the control were reported in Table 1 below.

A number of foam samples were obtained from BASF Aktiengesellschaft, Ludwigshafen, Germany. The samples were prepared in the same manner as the foam materials prepared in the examples of PCT WO01/55242. In general, the foam samples used in this example were prepared by reacting aliphatic isocyanates with polyetherols in the presence of a catalyst and complexed with an ethylenimine. The foam materials had the physical properties listed in Table 1. A blank in Table 1 indicates that no value was obtained for that particular property.

TABLE 1

| | Thickness | Density | | Delta Basis |
|---|---|---|---|---|
| Sample | (mm) | (g/cm³) | Basis Weight | Weight |
| Control* | 0.8 | 0.046 | 36 | — |
| s4f | 3.6 | 0.059 | 214 | −4 |
| s4d | 3.6 | 0.065 | 228 | 15 |
| L | 4.6 | 0.073 | 333 | — |
| G | 3.4 | 0.079 | 265 | — |
| T | 2.9 | 0.075 | 220 | −63 |
| O | 2.5 | 0.083 | 210 | −25 |

*Properties of H, BBA CD and BBA MD all were similar to Control.

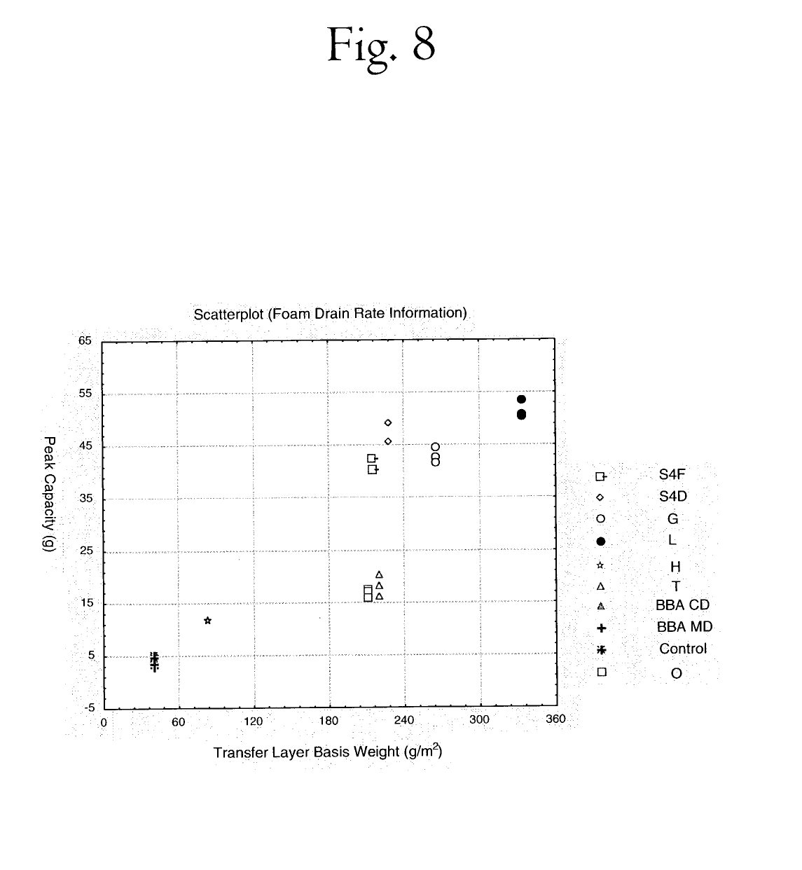

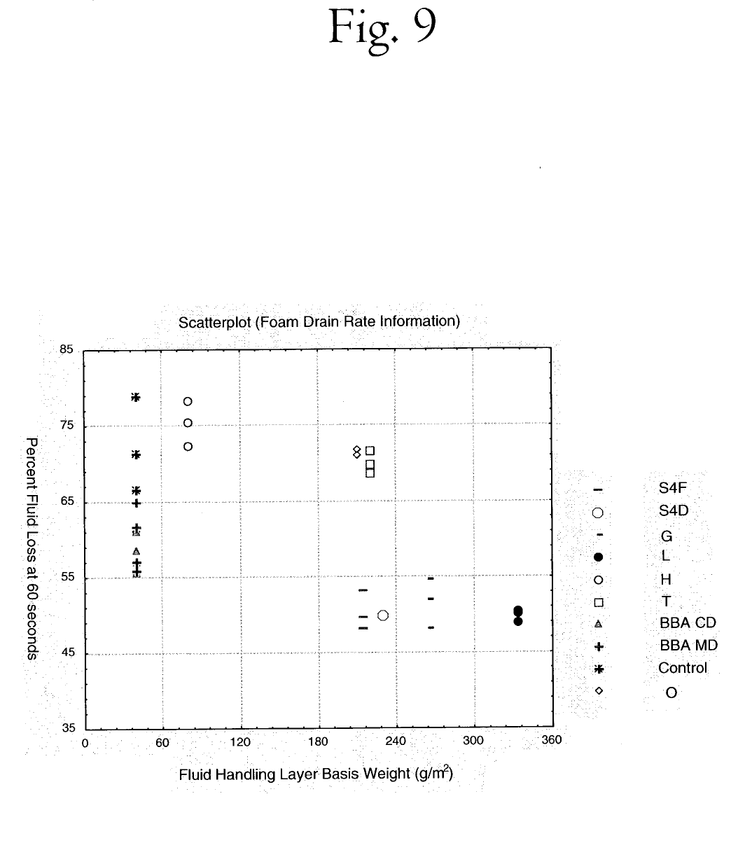

The respective fluid acquisition layers described above were subjected to the fluid drain rate test described herein. The maximum fluid held, or peak capacity, and the percent fluid loss after 60 seconds are shown in FIGS. 8 and 9, respectively. The data in the Figures represent the average of six sample values, whereby six samples of the same material were tested, and the average value (and standard deviation) calculated.

It is clear to see from FIGS. 8 and 9 that fluid acquisition layers made from foam materials in accordance with particularly preferred embodiments of the present invention provide for an acquisition layer losing less than about 55% of fluid after 60 seconds when subjected to the fluid drain rate test. In addition, the fluid acquisition layers, at a maximum, held more fluid than conventional fluid acquisition layers.

While the invention has been described with reference to particularly preferred embodiments and examples, those skilled in the art will appreciate that various modifications may be made thereto without departing significantly from the spirit and scope of the invention.

What is claimed is:

1. An absorbent article comprising:
a liquid impervious outer layer;
a liquid pervious inner layer;
a storage layer disposed between the outer layer and the inner layer; and
an acquisition layer disposed between the inner layer and the storage layer, whereby the acquisition layer has a fluid drain rate such that it loses less than 55% of fluid after 60 seconds.

2. The absorbent article of claim 1, wherein the acquisition layer comprises at least a polyurethane foam made from at least an aliphatic isocyanate.

3. The absorbent article of claim 1, wherein the fluid acquisition layer comprises at least one foam, the foam being comprised of at least one polymer selected from the group consisting of a polyurethane, a polyethylene, a polypropylene, a polyacrylic, a polyamide, a polyvinyl chloride, an epoxy, a polystyrene, a melamine-formaldehyde polymer, and combinations thereof.

4. The absorbent article of claim 1, wherein the fluid acquisition layer comprises at least one foam selected from a polyurethane polymer or a melamine-formaldehyde polymer.

5. The absorbent article of claim 3, wherein the polymer is a melamine-formaldehyde polymer.

6. The absorbent article of claim 3, wherein the foam has a density no greater than about 0.08 g/cc.

7. The absorbent article of claim 3, wherein the foam additionally comprises a stabilizing agent.

8. The absorbent article of claim 3, wherein the foam additionally comprises at least one additive selected from the group consisting of surfactants, fillers, additives, or combinations thereof.

9. The absorbent article of claim 8, wherein the additive is selected from the group consisting of a flame retardant, a reinforcing agent, an auxiliary blowing agent, a medicament, a fragrance, a colorant, a cleaner, an abrasive, and combinations thereof.

10. The absorbent article of claim 1, wherein the absorbent article is selected from the group consisting of a diaper, an incontinent brief, a training pant, a diaper holder, a diaper liner, a sanitary napkin, a hygienic garment, a swimming diaper, or combinations thereof.

11. The absorbent article of claim 2, wherein the aliphatic isocyanate is at least one isocyanate selected from the group consisting of hexamethylene diisocyanate, hexamethylene triisocyanate, bicycloheptane triisocyanate, undecanetriisocyanate, lysine ester triisocyanate, isophorone diisocyanate, dicyclohexylmethane diisocyanate, methylcyclohexane diisocyanate, dimethylcyclohexane diisocyanate, xylylene diisocyanate, tetramethylxylylene diisocyanate, their dimers, their trimers, and mixtures thereof.

12. The absorbent article of claim 11, wherein the aliphatic isocyanate is hexamethylene diisocyanate.

13. The absorbent article of claim 2, wherein the aliphatic isocyanate is reacted with a polyol.

14. The absorbent article of claim 13, wherein the polyol has a number average molecular weight of 100 to 5,000, and contains on the average 2 to 3 functional groups.

15. The absorbent article of claim 14, wherein the polyol has a number average molecular weight of from about 200 to about 3,000.

16. The absorbent article of claim 13, wherein the polyol is selected from the group consisting of polyether polyols, ethylene glycol, propylene glycol, glycerol, hexanetriol, triethanolamine, ethylene oxide, propylene oxide, butylene oxide, polytetramethylene ether glycol, lactone-type polyester polyols, polyol compounds obtained by condensing a dihydric or trihydric alcohol with s hydroxycarboxylic acid, polyol compounds obtained by condensing dicarboxylic acids with diols, condensed polyester polyols obtained by adding acid anhydrides with diols, and mixtures thereof.

17. The absorbent article of claim 16, wherein the polyol is a polyether polyol selected from the group consisting of polyethylene oxide, polypropylene oxide, poly(ethylene oxide-propylene oxide) copolymer, and mixtures thereof.

18. The absorbent article of claim 13, wherein the aliphatic isocyanate and polyol are reacted in the presence of a catalyst selected from the group consisting of alkali metal salts, lead salts, and alkaline earth metal salts of acetic acid, propionic acid, butyric acid, valeic acid, caproic acid, caprylic acid, capric acid, and 2-ethylhexanoic acid.

19. The absorbent article of claim 3, wherein the acquisition layer has a thickness within the range of from about 2.95 to about 7.0 mm.

20. The absorbent article of claim 3, wherein the foam has a basis weight within the range of from about 200 to about 500 g/m².

21. The absorbent article of claim 3, wherein the foam has a change in basis weight of less than about 30 g/m².

22. An absorbent article comprising:
a liquid impervious outer layer;
a liquid pervious inner layer;
a storage layer disposed between the outer layer and the inner layer; and
an acquisition layer disposed between the inner layer and the storage layer, whereby the absorbent article has an absorbent capacity under load in the front portion thereof of above about 1.1 g/cm², and an absorbent capacity under load in the rear portion thereof of between about 0.5 and about 0.9 g/cm².

23. The absorbent article of claim 22, wherein the acquisition layer comprises at least a polyurethane foam made from at least an aliphatic isocyanate.

24. The absorbent article of claim 22, wherein the fluid acquisition layer comprises at least one foam, the foam being comprised of at least one polymer selected from the group consisting of a polyurethane, a polyethylene, a polypropylene, a polyacrylic, a polyamide, a polyvinyl chloride, an epoxy, a polystyrene, a melamine-formaldehyde polymer, and combinations thereof.

25. The absorbent article of claim 22, wherein the acquisition layer has a fluid drain rate such that it loses less than 60% of fluid after 60 seconds.

26. The absorbent article of claim 25, wherein the acquisition layer has a fluid drain rate such that it loses less than 55% of fluid after 60 seconds.

27. The absorbent article of claim 24, wherein the at least one foam is comprised of at least a polyurethane polymer or a melamine-formaldehyde polymer.

28. The absorbent article of claim 27, wherein the polymer is a melamine-formaldehyde polymer.

29. The absorbent article of claim 24, wherein the foam has a density no greater than about 0.08 g/cc.

30. The absorbent article of claim 22, wherein the absorbent article is selected from the group consisting of a diaper, an incontinent brief, a training pant, a diaper holder, a diaper liner, a sanitary napkin, a hygienic garment, a swimming diaper, or combinations thereof.

31. The absorbent article of claim 24, wherein the acquisition layer has a thickness within the range of from about 2.95 to about 7.0 mm.

32. The absorbent article of claim 24, wherein the foam has a basis weight within the range of from about 200 to about 500 g/m².

33. The absorbent article of claim 24, wherein the foam has a change in basis weight of less than about 30 g/m².

34. An absorbent article comprising:
a liquid impervious outer layer;
a liquid pervious inner layer;
a storage layer disposed between the outer layer and the inner layer; and
an acquisition layer disposed between the inner layer and the storage layer, whereby the acquisition layer has a peak capacity of greater than about 25 grams.

35. The absorbent article of claim 34, wherein the acquisition layer comprises at least a polyurethane foam made from at least an aliphatic isocyanate.

36. The absorbent article of claim 34, wherein the fluid acquisition layer comprises at least one foam, the foam being comprised of at least one polymer selected from the group consisting of a polyurethane, a polyethylene, a polypropylene, a polyacrylic, a polyamide, a polyvinyl chloride, an epoxy, a polystyrene, a melamine-formaldehyde polymer, and combinations thereof.

37. The absorbent article of claim 34, wherein the fluid acquisition layer comprises at least one foam selected from a polyurethane polymer or a melamine-formaldehyde polymer.

38. The absorbent article of claim 34, wherein the foam has a density no greater than about 0.08 g/cc.

39. The absorbent article of claim 34, wherein the absorbent article is selected from the group consisting of a diaper, an incontinent brief, a training pant, a diaper holder, a diaper liner, a sanitary napkin, a hygienic garment, a swimming diaper, or combinations thereof.

40. The absorbent article of claim 36, wherein the acquisition layer has a thickness within the range of from about 2.95 to about 7.0 mm.

41. The absorbent article of claim 36, wherein the foam has a basis weight within the range of from about 200 to about 500 g/m².

42. The absorbent article of claim 36, wherein the foam has a change in basis weight of less than about 30 g/m².

43. The absorbent article of claim 34, wherein the acquisition layer has a peak capacity greater than about 35 grams.

44. A method of making an absorbent article comprising:
providing a liquid impervious outer layer;
providing a liquid impervious inner layer;
providing a storage layer;
disposing the storage layer between the inner layer and the outer layer;
providing an acquisition layer; and
disposing the acquisition layer between the inner layer and the storage layer, whereby the acquisition layer has a fluid drain rate such that it loses less than 55% of fluid after 60 seconds.

45. The method of claim 44, wherein the acquisition layer comprises at least a polyurethane foam made from at least an aliphatic isocyanate.

46. The method of claim 44, wherein the fluid acquisition layer comprises at least one foam, the foam being comprised of at least one polymer selected from the group consisting of a polyurethane, a polyethylene, a polypropylene, a polyacrylic, a polyamide, a polyvinyl chloride, an epoxy, a polystyrene, a melamine-formaldehyde polymer, and combinations thereof.

47. The method of claim 44, wherein the fluid acquisition layer comprises at least one foam selected from a polyurethane polymer or a melamine-formaldehyde polymer.

48. The method of claim 46, wherein the polymer is a melamine-formaldehyde polymer.

49. The method of claim 46, wherein the foam has a density no greater than about 0.08 g/cc.

50. The method of claim 46, wherein the acquisition layer has a thickness within the range of from about 2.95 to about 7.0 mm.

51. The method of claim 46, wherein the foam has a basis weight within the range of from about 200 to about 500 g/m².

52. The method of claim 46, wherein the foam has a change in basis weight of less than about 30 g/m².

53. The method of claim 44, wherein the acquisition layer has a peak capacity of greater than about 25 grams of fluid.

54. The method of claim 53, wherein the peak capacity is greater than about 30 grams of fluid.

55. The method of claim 46, wherein the foam acquisition layer is prepared by reacting an aliphatic isocyanate with a polyol, whereby the aliphatic isocyanate is at least one isocyanate selected from the group consisting of hexamethylene diisocyanate, hexamethylene triisocyanate, bicycloheptane triisocyanate, undecanetriisocyanate, lysine ester triisocyanate, isophorone diisocyanate, dicyclohexylmethane diisocyanate, methylcyclohexane diisocyanate, dimethylcyclohexane diisocyanate, xylylene diisocyanate, tetramethylxylylene diisocyanate, their dimers, their trimers, and mixtures thereof.

56. The method of claim 55, wherein the aliphatic isocyanate is hexamethylene diisocyanate.

57. The method of claim 55, wherein the polyol has a number average molecular weight of 100 to 5,000, and contains on the average 2 to 3 functional groups.

58. The method of claim 57, wherein the polyol has a number average molecular weight of from about 200 to about 3,000.

59. The absorbent article of claim 55, wherein the polyol is selected from the group consisting of polyether polyols, ethylene glycol, propylene glycol, glycerol, hexanetriol, triethanolamine, ethylene oxide, propylene oxide, butylene oxide, polytetramethylene ether glycol, lactone-type polyester polyols, polyol compounds obtained by condensing a dihydric or trihydric alcohol with s hydroxycarboxylic acid, polyol compounds obtained by condensing dicarboxylic acids with diols, condensed polyester polyols obtained by adding acid anhydrides with diols, and mixtures thereof.

60. The absorbent article of claim 59, wherein the polyol is a polyether polyol selected from the group consisting of polyethylene oxide, polypropylene oxide, poly(ethylene oxide-propylene oxide) copolymer, and mixtures thereof.

61. A method of making an absorbent article comprising:
providing a liquid impervious outer layer;
providing a liquid impervious inner layer;
providing a storage layer;
disposing the storage layer between the inner layer and the outer layer;
providing an acquisition layer; and
disposing the acquisition layer between the inner layer and the storage layer,
whereby the absorbent article has an absorbent capacity under load in the front portion thereof of above about 1.1 g/cm², and an absorbent capacity under load in the rear portion thereof of between about 0.5 and about 0.9 g/cm².

62. The method of claim 61, wherein the acquisition layer comprises at least a polyurethane foam made from at least an aliphatic isocyanate.

63. The method of claim 61, wherein the fluid acquisition layer comprises at least one foam, the foam being comprised of at least one polymer selected from the group consisting of a polyurethane, a polyethylene, a polypropylene, a polyacrylic, a polyamide, a polyvinyl chloride, an epoxy, a polystyrene, a melamine-formaldehyde polymer, and combinations thereof.

64. The method of claim 61, wherein the fluid acquisition layer comprises at least one foam selected from a polyurethane polymer or a melamine-formaldehyde polymer.

65. The method of claim 63, wherein the polymer is a melamine-formaldehyde polymer.

66. The method of claim 63, wherein the foam has a density no greater than about 0.08 g/cc.

67. The method of claim 63, wherein the acquisition layer has a thickness within the range of from about 2.95 to about 7.0 mm.

68. The method of claim 63, wherein the foam has a basis weight within the range of from about 200 to about 500 g/m².

69. The method of claim 63, wherein the foam has a change in basis weight of less than about 30 g/m².

70. The method of claim 61, wherein the acquisition layer has a peak capacity of greater than about 15 grams of fluid.

71. The method of claim 70, wherein the peak capacity is greater than about 30 grams of fluid.

72. The method of claim 63, wherein the foam acquisition layer is prepared by reacting an aliphatic isocyanate with a polyol, whereby the aliphatic isocyanate is at least one isocyanate selected from the group consisting of hexamethylene diisocyanate, hexamethylene triisocyanate, bicycloheptane triisocyanate, undecanetriisocyanate, lysine ester triisocyanate, isophorone diisocyanate, dicyclohexylmethane diisocyanate, methylcyclohexane diisocyanate, dimethylcyclohexane diisocyanate, xylylene diisocyanate, tetramethylxylylene diisocyanate, their dimers, their trimers, and mixtures thereof.

73. The method of claim 72, wherein the aliphatic isocyanate is hexamethylene diisocyanate.

74. The method of claim 72, wherein the polyol has a number average molecular weight of 100 to 5,000, and contains on the average 2 to 3 functional groups.

75. The method of claim 74, wherein the polyol has a number average molecular weight of from about 200 to about 3,000.

76. The absorbent article of claim 72, wherein the polyol is selected from the group consisting of polyether polyols, ethylene glycol, propylene glycol, glycerol, hexanetriol, triethanolamine, ethylene oxide, propylene oxide, butylene oxide, polytetramethylene ether glycol, lactone-type polyester polyols, polyol compounds obtained by condensing a dihydric or trihydric alcohol with s hydroxycarboxylic acid, polyol compounds obtained by condensing dicarboxylic acids with diols, condensed polyester polyols obtained by adding acid anhydrides with diols, and mixtures thereof.

77. The absorbent article of claim 76, wherein the polyol is a polyether polyol selected from the group consisting of polyethylene oxide, polypropylene oxide, poly(ethylene oxide-propylene oxide) copolymer, and mixtures thereof.

78. A method of making an absorbent article comprising:
providing a liquid impervious outer layer;
providing a liquid impervious inner layer;
providing a storage layer;
disposing the storage layer between the inner layer and the outer layer;
providing an acquisition layer; and
disposing the acquisition layer between the inner layer and the storage layer,
whereby acquisition layer has a peak capacity of greater than about 25 grams.

79. The method of claim 78, wherein the acquisition layer comprises at least a polyurethane foam made from at least an aliphatic isocyanate.

80. The method of claim 78, wherein the fluid acquisition layer comprises at least one foam, the foam being comprised of at least one polymer selected from the group consisting of a polyurethane, a polyethylene, a polypropylene, a polyacrylic, a polyamide, a polyvinyl chloride, an epoxy, a polystyrene, a melamine-formaldehyde polymer, and combinations thereof.

81. The method of claim 78, wherein the fluid acquisition layer comprises at least one foam selected from a polyurethane polymer or a melamine-formaldehyde polymer.

82. The method of claim 80, wherein the foam has a density no greater than about 0.08 g/cc.

83. The method of claim 80, wherein the acquisition layer has a thickness within the range of from about 2.95 to about 7.0 mm.

84. The method of claim 80, wherein the foam has a basis weight within the range of from about 200 to about 500 g/m².

85. The method of claim 80, wherein the foam has a change in basis weight of less than about 30 g/m².

86. The method of claim 78, wherein the acquisition layer has a peak capacity of greater than about 30 grams of fluid.

87. The method of claim 78, wherein the peak capacity is greater than about 35 grams of fluid.
